(19) United States
(12) Patent Application Publication
(10) Pub. No.: US 2016/0046987 A1
(43) Pub. Date: Feb. 18, 2016

(54) LIBRARY GENERATION FOR NEXT-GENERATION SEQUENCING

(71) Applicant: ABBOTT MOLECULAR INC., Des Plaines, IL (US)

(72) Inventors: Dae Hyun Kim, Des Plaines, IL (US); Marc Henry Domanus, Des Plaines, IL (US)

(21) Appl. No.: 14/826,904

(22) Filed: Aug. 14, 2015

Related U.S. Application Data
(60) Provisional application No. 62/037,327, filed on Aug. 14, 2014.

(51) Int. Cl. C12Q 1/68

(52) U.S. Cl. CPC C12Q 1/6874

(57) ABSTRACT

Provided herein is technology relating to next-generation sequencing (NGS) and particularly, but not exclusively, to methods and compositions for preparing NGS libraries, e.g., to prepare NGS libraries for use in a NGS workflow.

### CROSS-REFERENCE TO RELATED APPLICATIONS

The present application claims priority to U.S. Provisional Application Ser. No. 62/037,327 filed Aug. 14, 2014, the entirety of which is incorporated by reference herein.

### FIELD OF TECHNOLOGY

Provided herein is technology relating to next-generation sequencing (NGS) and particularly, but not exclusively, to methods and compositions for preparing NGS libraries, e.g., to prepare NGS libraries for use in a NGS workflow.

### BACKGROUND

Next generation sequencing platforms generally require as input a specific concentration of a nucleic acid library to be loaded onto the sequencer workflow for clonal amplification. The sequence output depends on the initial concentration—loading too low of a concentration of the NGS library results in low sequencer output while loading too high of a concentration of the NGS library results in low quality sequence, unusable sequencer output, or no sequencer output.

Some conventional solutions have been designed for DNA concentration, size selection, purification, and normalization for NGS. Common normalization approaches include direct quantification, e.g., by spectrophotometry, fluorimetry, quantitative PCR, or electrophoresis, followed by calculation of desired concentrations and dilution of samples to a normalized concentration. Other conventional solutions include kits sold by Life Technologies, Illumina, Invitrogen, and Corning/AxyPrep for preparing amplicon libraries for sequencing. However, these solutions involve lengthy amounts of time, are associated with multiple hands-on steps, and are often compatible only with a specific NGS platform. In particular, the Life Technologies Ion Torrent™ Ion Ampliseg™ Library Preparation protocol comprises a library equalization step before sequencing (e.g., through use of an Ion Library Equalizer™ kit). This step requires the NGS amplicon library to be amplified further in the presence of Ion Equalizer™ Primers during a 7-cycle PCR. This step adds both total time and user hands-on time to the sample preparation procedure and, furthermore, the method is specific to the Ion Torrent sequencing platform. In addition, the Illumina TruSeq™ Custom Amplicon Library Preparation protocol requires multiple steps and considerable time input by a user (e.g., a total duration of 1 hour and 20 minutes with 30 minutes of hands-on time). The Illumina library normalization procedure is performed after final NGS amplicon library cleanup and size selection and it is specific for Illumina formatted Truseq™ amplicon libraries. Technology provided by Invitrogen in the SequalPrep™ Normalization product purifies DNA in a size range of 100 bp to 20 kbp and has a recommended input of at least 250 ng of nucleic acid product. Similarly, the Corning/Axygen Biosciences AxyPrep Mag™ Normalizer product is similarly configured for recoveries of 100 ng or more DNA.

Methods that require multiple pipetting steps and use of multiple vessels have greater opportunities for introduction of errors. In addition, costs in time and resources are associated with procedures having many steps. Consequently, there is a need for new technologies to normalize nucleic acid libraries for next-generation sequencing that are simple, require few steps, and are generally applicable to multiple next-generation sequencing platforms. In addition, there is a need for normalization technologies that are applicable to samples having less than 100 ng amounts of product, such as amplicon panels produced from low-cycle number amplification used to retain coverage uniformity of sequencing targets.

### SUMMARY

The technology provided herein simplifies next generation sequencing workflows by alleviating the need to quantify or concentration normalize NGS libraries prior to NGS sequencer workflow loading. The technology described provides a NGS workflow having fewer steps, less hands-on time, and less turnaround time than conventional technologies. The technology is generic to any library prepared for NGS. For example, in some embodiments the technology is used to process an NGS amplicon panel library. Embodiments of the methods use a reduced number of tube transfers and pipetting steps and have a reduced cost compared to conventional NGS amplicon methods. In particular, hands-on time and sample turnaround time are reduced relative to conventional technologies by eliminating some steps of conventional technologies such as purification, size selection, and direct quantification (e.g., by spectroscopy, fluorimetry, quantitative PCR, and electrophoresis) that are performed in conventional technologies prior to the library being ready for the sequencer workflow. In some embodiments, NGS libraries are ready for NGS sequencer workflow (e.g., clonal amplification) loading without further dilution, purification, or quantification.

Accordingly, provided herein are embodiments of technology relating to a method for normalizing the concentration of an NGS library, the method comprising mixing a next-generation sequencing library comprising a first amount of library fragments with a capture substrate having a capacity to bind a second amount of library fragments that is less than the first amount of library fragments to provide a capture mixture comprising unbound library fragments and a capture substrate comprising bound library fragments; and eluting the bound library fragments from the capture substrate to provide a concentration normalized NGS library comprising library fragments. In some embodiments, the methods further comprise binding the library fragments to the capture substrate. Further embodiments comprise steps such as removing the unbound library fragments from the capture mixture, washing the capture substrate comprising the bound library fragments, and/or ligating an adapter to a library fragment. The technology is not limited in the type of capture substrate that is used; e.g., in some embodiments the capture substrate comprises a paramagnetic microparticle functionalized with a carboxyl group (COOH/COO—) an amine group, a metal ion, an encapsulated carboxyl group, silica (SiOH), diethyl aminoethyl, or a group that hybridizes to a nucleic acid sequence (e.g., a complementary sequence).

The technology finds use in providing a concentration normalized NGS library having a defined amount of library fragments. In some embodiments, the ratio of the first amount of library fragments (e.g., in the next-generation sequencing library) to the second amount of library fragments (e.g., in the concentration normalized NGS library) is more than 1000, more than 100, or more than 10.

The technology provides for the size selection of a NGS library; thus, in some embodiments methods comprise size-selecting the NGS library by adjusting buffer components (e.g., salts (e.g., sodium chloride (NaCl), lithium chloride (LiCl), barium chloride (BaCl₂), potassium chloride (KCl), calcium chloride (CaCl₂), magnesium chloride (MgCl₂), and cesium chloride (CsCl) at approximately 0.005 M to approximately 5 M; e.g., at approximately 0.1 M to approximately 0.5 M; at approximately 0.15 M to approximately 0.4 M; or at approximately 2 M to approximately 4 M), precipitating reagents, crowding reagents (e.g., 5% to 20% PEG, e.g., 5%, 6%, 7%, 8%, 9%, 10%, 11%, 12%, 13%, 14%, 15%, 16%, 17%, 18%, 19%, 20% PEG; e.g., 5%, 6%, 7%, 8%, 9%, 10%, 11%, 12%, 13%, 14%, 15%, 16%, 17%, 18%, 19%, 20% PEG having an average molecular weight of from 250 to approximately 10,000; from approximately 1000 to approximately 10,000; from approximately 2500 to approximately 10,000; from approximately 6000 to approximately 10,000; from approximately 6000 to approximately 8000; from approximately 7000 to approximately 9000; from approximately 8000 to approximately 10,000) and some embodiments comprise size-selecting the NGS library by adjusting ionic strength.

Furthermore, the technology finds use in providing a multiplex NGS library (e.g., comprising two or more normalized NGS libraries representing, e.g., two or more subjects, two or more samples, two or more patients, two or more genes, two or more assays, etc.) for loading on a sequencing apparatus. Thus, the technology provides an efficient method for increasing the throughput and efficiency of genetic and/or genomic analysis by sequencing (e.g., NGS). Accordingly, in some embodiments, methods further comprise combining two or more concentration normalized NGS libraries to provide a multiplex concentration normalized NGS library.

In some embodiments, the methods further comprise adding a nucleic acid precipitating reagent or a crowding reagent, e.g., to promote binding of the nucleic acid (e.g., library fragments) to the capture substrate.

The methods provided are generally applicable to providing a concentration normalized NGS library (or multiplexed mixture of NGS libraries) for NGS platforms. Particular embodiments are advantageous in providing concentration normalized NGS libraries having particular concentrations or amounts of DNA or DNA having particular fragment lengths. Some embodiments find use in concentration normalization of input NGS libraries having particular concentrations and/or amounts of DNA. For instance, some embodiments provide a concentration normalized NGS library comprising DNA at a concentration of less than 1 nM, less than 0.75 nM, less than 0.55 nM, or less than 0.25 nM. Some embodiments provide a concentration normalized NGS library comprising DNAs that comprise more than 100 bp. Some embodiments provide a concentration normalized NGS library comprising less than 200, less than 150, less than 100, less than 50, less than 25, less than 10, and/or less than 5 amplicons. Some embodiments find use in normalizing a next-generation sequencing library (e.g., as input to the methods) that comprises less than 250 ng, less than 200, less than 150, or less than 100 ng of DNA.

The technology provides particular advantages with respect to decreasing time for normalization and/or hands-on time and/or cost. For example, in some embodiments the steps of the method are performed in a single vessel (e.g., sample tube, single well, etc.). Further, in some embodiments the technology does not depend on the use of any particular sequence (e.g., the technology does not use a sequence-based capture probe) and the technology is not specific to any particular NGS platform.

Some embodiments provide a method for normalizing the concentration of a NGS library, the method consisting of, comprising, or consisting essentially of mixing a next-generation sequencing library comprising a first amount of library fragments with a capture substrate (e.g., comprising a paramagnetic microparticle functionalized with a carboxyl group) having a capacity to bind a second amount of library fragments that is less than the first amount of library fragments to provide a capture mixture comprising unbound library fragments and a capture substrate comprising bound library fragments. In some embodiments, a subsequent step comprises eluting the bound library fragments from the capture substrate to provide a concentration normalized NGS library. In some embodiments, the methods further comprise steps that occur after the mixing step and before the eluting step such as: removing the unbound library fragments from the capture mixture; washing the capture substrate comprising the bound library fragments; size-selecting the NGS library (e.g., by adjusting buffer components and/or adjusting ionic strength); and/or adding a nucleic acid precipitating reagent (e.g., PEG). In some embodiments, the methods comprise steps that occur after the eluting step such ligating an adapter to a library fragment and/or combining two or more concentration normalized NGS libraries to provide a multiplex concentration normalized NGS library.

In particular embodiments, the methods comprise mixing a next-generation sequencing library comprising a first amount of library fragments with a capture substrate (e.g., comprising a paramagnetic microparticle functionalized with a carboxyl group) having a capacity to bind a second amount of library fragments that is less than the first amount of library fragments to provide a capture mixture comprising unbound library fragments and a capture substrate comprising bound library fragments, wherein the ratio of the first amount of library fragments to the second amount of library fragments is more than 1000, more than 100, or more than 10; wherein the concentration normalized NGS library comprises DNA at a concentration of less than 1 nM, less than 0.75 nM, less than 0.55 nM, or less than 0.25 nM; wherein the concentration normalized NGS library comprises DNAs that comprise more than 100 bp; wherein the concentration normalized NGS library comprises less than 200, less than 150, less than 100, less than 50, less than 25, less than 10, or less than 5 library fragments; wherein the next-generation sequencing library comprises less than 250 ng, less than 200, less than 150, or less than 100 ng of DNA; and/or wherein the steps of the method are performed in a single vessel.

In some embodiments, the technology provides a method for normalizing the concentration of a NGS library by mixing a NGS library comprising a first amount of library fragments with a capture substrate having a capacity to bind a second amount of library fragments that is less than the first amount of library fragments to provide a capture mixture comprising unbound library fragments and a capture substrate comprising bound library fragments; and eluting the bound library fragments from the capture substrate to provide a concentration normalized NGS library, wherein the concentration normalized NGS library comprises DNA at a concentration of less than 1 nM, less than 0.75 nM, less than 0.55 nM, or less than 0.25 nM; the concentration normalized NGS library comprises DNAs that comprise more than 100 bp; the concentration normalized NGS library comprises less than 200, less than 150, less than 100, less than 50, less than 25, less than 10, or less than 5 amplicons; and/or the input NGS library comprises less than 250 ng, less than 200, less than 150, or less than 100 ng of DNA.

Related embodiments of the technology provide a method for sequencing a nucleic acid comprising a method for library generation as described herein (e.g., a method for generating a concentration normalized NGS library) and further comprising loading the concentration normalized NGS library into a next generation sequencer work flow.

In addition, embodiments are described relating to a concentration normalized NGS library, e.g., as produced by a method described herein. For example, in some embodiments the technology provides a composition comprising a concentration normalized NGS library comprising one or more library fragments, e.g., library fragments comprising sequences from regions of interest (e.g., from nucleic acid sequence targets to be sequenced). In some embodiments, the library fragments are linked to and/or comprise adapters for sequencing (e.g., sequence-platform specific adapters). In some embodiments, the technology provides a composition comprising library fragments having a length greater than 75 bp or bases, greater than 80 bp or bases, greater than 85 bp or bases, greater than 90 bp or bases, greater than 95 bp or bases, greater than 100 bp or bases, greater than 105 bp or bases, greater than 110 bp or bases, greater than 115 bp or bases, greater than 120 bp or bases; e.g., in some embodiments, the composition does not comprise library fragments of approximately 100 bp or bases or shorter.

In some embodiments, the composition comprises a number of library fragments that is less than 500 library fragments, less than 450 library fragments, less than 400 library fragments, less than 350 library fragments, less than 300 library fragments, less than 250 library fragments, less than 200 library fragments, less than 150 library fragments, less than 100 library fragments, less than 50 library fragments, less than 25 library fragments, e.g., in some embodiments, the technology provides a composition comprising 1 to 150 library fragments. In some embodiments, the technology provides a composition comprising nucleic acids (e.g., a NGS library) having a concentration less than 1 nM, e.g., less than 0.90 nM, less than 0.80 nM, less than 0.70 nM, less than 0.60 nM, less than 0.55 nM, less than 0.50 nM, less than 0.45 nM, less than 0.40 nM, less than 0.35 nM, less than 0.30 nM, less than 0.25 nM, less than 0.20 nM, less than 0.15 nM, or less than 0.10 nM.

In some embodiments, the technology is related to a composition comprising a concentration normalized NGS library comprising one or more library fragments, e.g., library fragments comprising sequences from regions of interest of a nucleic acid to be sequenced, and further comprising a capture substrate, e.g., a non-specific capture substrate, e.g., a magnetic particle comprising silica and/or a functional group coated surface, e.g., a magnetic particle comprising a carboxyl group (COOH/COO—). In some embodiments, the magnetic particle comprises an amine group, a metal ion, an encapsulated carboxyl group, silica (SiOH), diethyl aminoethyl, or a group that hybridizes to a nucleic acid sequence (e.g., a complementary sequence). In some embodiments, the technology is related to a composition comprising a concentration normalized NGS library comprising one or more library fragments, e.g., library fragments comprising sequences from regions of interest of nucleic acid to be sequenced, and further comprising a capture substrate, e.g., a non-specific capture substrate, e.g., a magnetic particle comprising a functional group coated surface, e.g., a magnetic particle comprising free COO—/COOH groups and further comprising a buffer, e.g., one or more nucleic acid precipitating agent(s), e.g., PEG, and, in some embodiments, a salt (e.g., NaCl), Tris-HCl, and/or citrate.

In some embodiments, the technology is related to a composition comprising a concentration normalized NGS library comprising one or more library fragments, e.g., library fragments comprising sequences from regions of interest of a nucleic acid to be sequenced, and further comprising a buffer that elutes and/or stabilizes the nucleic acids of the concentration normalized NGS library, e.g., a buffered salt solution, e.g., comprising Tris-HCl, EDTA, and a cation (e.g., from 0.1 M to 0.5 M).

Some embodiments provide a composition comprising a normalized NGS library (e.g., ready for loading into a NGS sequencing workflow) as described herein (e.g., that does not comprise nucleic acids less than 100 bases or by in length), a polymerase, and nucleotides (e.g., labeled nucleotides) to provide, e.g., a sequencing reaction mixture. For example, in some embodiments the technology relates to a composition comprising library fragments of a NGS library (e.g., comprising one or more adapters) at a library fragment concentration of less than 1 nM (e.g., less than 0.5 nM) and/or comprising less than 500 library fragments. Some embodiments provide a composition comprising a normalized NGS library that does not comprise a diluent (e.g., to adjust the concentration for loading into a NGS sequencing workflow as described herein), a polymerase, and nucleotides (e.g., labeled nucleotides) to provide, e.g., a sequencing reaction mixture. For example, in some embodiments the technology relates to a composition comprising library fragments of a NGS library (e.g., comprising one or more adapters) at library fragment concentration of less than 1 nM (e.g., less than 0.5 nM) and/or comprising less than 500 library fragments and that does not comprise a diluent added to adjust the concentration.

Some embodiments provide kits for producing a concentration normalized NGS library. For example, some embodiments provide a capture substrate (e.g., a non-specific capture substrate, e.g., a magnetic capture substrate comprising silica and/or free COO—/COOH groups) having a binding capacity for nucleic acids that is less than 250 ng and/or less than 100 ng (e.g., less than 200 ng, less than 150 ng, less than 100 ng, less than 75 ng, less than 50 ng, less than 25 ng, and/or less than 10 ng) and one or more of a binding buffer (e.g., comprising a nucleic acid precipitating reagent such as a polyalcohol (e.g., PEG) and/or a crowding agent (e.g., PVP)), a wash buffer (e.g., comprising a detergent, a salt such as NaCl, and/or an alcohol (e.g., ethanol)), and/or an elution buffer.

Some embodiments provide kits for producing a concentration normalized NGS library. For example, some embodiments provide a capture substrate (e.g., a non-specific capture substrate, e.g., a capture substrate (e.g., a magnetic particle) comprising a carboxyl group (COOH/COO—) having a binding capacity for nucleic acids that is less than 250 ng and/or less than 100 ng (e.g., less than 200 ng, less than 150 ng, less than 100 ng, less than 75 ng, less than 50 ng, less than 25 ng, and/or less than 10 ng) and one or more of a binding buffer (e.g., comprising a nucleic acid precipitating reagent such as a polyalcohol (e.g., PEG) and/or a crowding agent (e.g., PVP)), a wash buffer (e.g., comprising a detergent, a salt such as NaCl, and/or an alcohol (e.g., ethanol)), and/or an elution buffer. In some embodiments, the magnetic particle comprises an amine group, a metal ion, an encapsulated carboxyl group, silica (SiOH), diethyl aminoethyl, or a group that hybridizes to a nucleic acid sequence (e.g., a complementary sequence).

Some embodiments provide a kit for sequencing a nucleic acid (e.g., on a NGS sequencing platform). For example, some embodiments provide a capture substrate (e.g., a non-specific capture substrate, e.g., a magnetic capture substrate comprising silica and/or comprising free COO—/COOH groups) having a binding capacity for nucleic acids that is less than 250 ng and/or less than 100 ng (e.g., less than 200 ng, less than 150 ng, less than 100 ng, less than 75 ng, less than 50 ng, less than 25 ng, and/or less than 10 ng) and/or a composition comprising a capture substrate having a binding capacity for nucleic acids that is less than 250 ng and/or less than 100 ng (e.g., less than 200 ng, less than 150 ng, less than 100 ng, less than 75 ng, less than 50 ng, less than 25 ng, and/or less than 10 ng); one or more of a binding buffer (e.g., comprising a nucleic acid precipitating reagent such as a polyalcohol (e.g., PEG) and/or a crowding agent (e.g., PVP)), a wash buffer (e.g., comprising a detergent, a salt such as NaCl, and/or an alcohol (e.g., ethanol)), and/or an elution buffer; a polymerase; adapter oligonucleotides (in some embodiments, the kits further comprise a ligase for ligating the adapters to the amplicons); and/or nucleotides (e.g., labeled nucleotides).

Some embodiments provide a system for producing a concentration normalized NGS library. Examples of system embodiments comprise a capture substrate (e.g., a non-specific capture substrate, e.g., a magnetic capture substrate comprising silica and/or free COO—/COOH groups) having a binding capacity for nucleic acids that is less than 250 ng and/or less than 100 ng (e.g., less than 200 ng, less than 150 ng, less than 100 ng, less than 75 ng, less than 50 ng, less than 25 ng, and/or less than 10 ng); one or more of a binding buffer (e.g., comprising a nucleic acid precipitating reagent such as a polyalcohol (e.g., PEG) and/or a crowding agent (e.g., PVP)), a wash buffer (e.g., comprising a detergent, a salt such as NaCl, and/or an alcohol (e.g., ethanol)), and/or an elution buffer; and further include, in some embodiments, a magnet.

Some embodiments provide a system for sequencing a nucleic acid. For example, some embodiments comprise a capture substrate (e.g., a non-specific capture substrate, e.g., a magnetic capture substrate comprising silica and/or free COO—/COOH groups) having a binding capacity for nucleic acids that is less than 250 ng and/or less than 100 ng (e.g., less than 200 ng, less than 150 ng, less than 100 ng, less than 75 ng, less than 50 ng, less than 25 ng, and/or less than 10 ng); one or more of a binding buffer (e.g., comprising a nucleic acid precipitating reagent such as a polyalcohol (e.g., PEG) and/or a crowding agent (e.g., PVP)), a wash buffer (e.g., comprising a detergent, a salt such as NaCl, and/or an alcohol (e.g., ethanol)), and/or an elution buffer; and further include, in some embodiments, a magnet, adapter oligonucleotides (and, in some embodiments, a ligase), a polymerase, nucleotides, a sequencing apparatus, a computer for controlling the sequencing apparatus and/or for collecting and analyzing sequencing data, and computer software to provide instructions to the computer and/or the sequencing apparatus. Some embodiments further comprise one or more machines and/or automated apparatuses for liquid handling, sample manipulation, movement and tracking of samples, etc. For example, in some embodiments, an automated machine (e.g., performing instructions provided by software and/or connected to a computer) performs one or more steps such as: providing a fragment library, formatting the fragment library for next generation sequencing (e.g., comprising ligating/attaching adapters), combining the formatted fragment library with a defined recovery-limiting type and/or amount of capture substrate (e.g., carboxyl-modified magnetic beads), preferentially binding to the capture substrate library fragments of a desired size range relative to library fragments outside the desired size range (e.g., by using buffer conditions (e.g., salt concentrations and/or pH) that promote binding of library fragments of the desired size range to the capture substrate and that do not promote binding of library fragments outside of the desired size range to the capture substrate), capturing bound library fragments (e.g., using a magnet), removing excess unbound library fragments, washing bound library fragments, eluting bound library fragments, collecting eluted library fragments, diluting eluted library fragments, and sequencing eluted library fragments.

In some embodiments, simultaneous size selection, purification, and concentration normalization of a DNA amplicon library is performed by mixing (e.g., in a 1:2 ratio) a sample comprising a DNA amplicon library with a solution comprising PEG 8000 (e.g., 20% PEG 8000), NaCl (e.g., 0.5 M NaCl), and 8-μm magnetic beads functionalized with carboxylate groups (e.g., at 5% w/v beads/solution); washing the beads with 60% EtOH, and eluting the DNA amplicon library from the beads to prepare a size selected, purified, and concentration normalized DNA amplicon library ready for input to a NGS workflow. In some embodiments, the concentration normalized DNA amplicon library comprised a concentration of DNA that is 0.2 nM to 0.3 nM. In some embodiments, the concentration normalized DNA amplicon library comprises DNA that is greater than approximately 100 base pairs.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a plot showing that bead quantity limits the quantity of DNA recovered.

FIG. 2 is a plot showing the concentration of NGS amplicon-based libraries before capture/normalization (left column of each pair (with hashing fill) for each sample) and after capture/normalization (right column of each pair (with solid fill) for each sample) according to embodiments of the technology provided herein.

FIG. 3 is a plot showing the size distribution of NGS amplicon based libraries before and after capture/normalization according to embodiments of the technology provided herein.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to NGS and particularly, but not exclusively, to methods and compositions for preparing NGS libraries ready for use in a NGS workflow. In the description of the technology herein, the section headings used herein are for organizational purposes only and are not to be construed as limiting the described subject matter in any way; for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleic acid” shall mean any nucleic acid molecule, including, without limitation, DNA, RNA, and hybrids thereof. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term as used herein also encompasses cDNA, that is complementary, or copy, DNA produced from an RNA template, for example by the action of a reverse transcriptase.

As used herein, “nucleic acid sequencing data”, “nucleic acid sequencing information”, “nucleic acid sequence”, “genomic sequence”, “genetic sequence”, “fragment sequence”, or “nucleic acid sequencing read” denotes any information or data that is indicative of the order of the nucleotide bases (e.g., adenine, guanine, cytosine, and thymine/uracil) in a molecule (e.g., a whole genome, a whole transcriptome, an exome, oligonucleotide, polynucleotide, fragment, etc.) of DNA or RNA.

It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

Reference to a base, a nucleotide, or to another molecule may be in the singular or plural. That is, “a base” may refer to a single molecule of that base or to a plurality of the base, e.g., in a solution.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, the term “target nucleic acid” or “target nucleotide sequence” refers to any nucleotide sequence (e.g., RNA or DNA), the manipulation of which may be deemed desirable for any reason by one of ordinary skill in the art. In some embodiments, “target nucleic acid” refers to a nucleotide sequence whose nucleotide sequence is to be determined or is desired to be determined. In some embodiments, the term “target nucleotide sequence” refers to a sequence to which a partially or completely complementary primer or probe is generated.

As used herein, the term “region of interest” refers to a nucleic acid that is analyzed (e.g., using one of the compositions, systems, or methods described herein). In some embodiments, the region of interest is a portion of a genome or region of genomic DNA (e.g., comprising one or chromosomes or one or more genes). In some embodiments, mRNA expressed from a region of interest is analyzed. In some embodiments, the region of interest is a region, locus, portion, etc. of a nucleic acid.

As used herein, the term “corresponds to” or “corresponding” is used in reference to a contiguous nucleic acid or nucleotide sequence (e.g., a subsequence) that is complementary to, and thus “corresponds to”, all or a portion of a target nucleic acid sequence.

As used herein, the phrase “a clonal plurality of nucleic acids” refers to the nucleic acid products that are complete or partial copies of a template nucleic acid from which they were generated. These products are substantially or completely or essentially identical to each other, and they are complementary copies of the template nucleic acid strand from which they are synthesized, assuming that the rate of nucleotide misincorporation during the synthesis of the clonal nucleic acid molecules is 0%.

As used herein, the term “library” refers to a plurality of nucleic acids, e.g., a plurality of different nucleic acids. In some embodiments, a “library” is a “library panel” or an “amplicon library panel”. As used herein, an “amplicon library panel” is a collection of amplicons that are related, e.g., to a disease (e.g., a polygenic disease), disease progression, developmental defect, constitutional disease (e.g., a state having an etiology that depends on genetic factors, e.g., a heritable (non-neoplastic) abnormality or disease), metabolic pathway, pharmacogenomic characterization, trait, organism (e.g., for species identification), group of organisms, geographic location, organ, tissue, sample, environment (e.g., for metagenomic and/or ribosomal RNA (e.g., ribosomal small subunit (SSU), ribosomal large subunit (LSU), 5S, 16S, 18S, 23S, 28S, internal transcribed sequence (ITS) rRNA) studies), gene, chromosome, etc. For example, a cancer amplicon panel may comprise a set of primers for use in sequencing hundreds, thousands, or more loci, regions, genes, single nucleotide polymorphisms, alleles, markers, etc. that are associated with cancer. In some embodiments, an amplicon library panel provides for highly multiplexed and targeted resequencing, e.g., to detect mutations associated with disease. In some embodiments, a “library” comprises a plurality (e.g., collection) of “library fragments”; a “library fragment” is a nucleic acid. In some embodiments, library fragments are produced by fragmenting a larger nucleic acid, e.g., physical (e.g., shearing), enzymatic (e.g., by nuclease), and/or chemical treatment. In some embodiments, library fragments are produced by amplification (e.g., PCR) and are thus amplicons corresponding to and/or derived from a nucleic acid (e.g., a nucleic acid to be sequenced).

As used herein, a “subsequence” of a nucleotide sequence refers to any nucleotide sequence contained within the nucleotide sequence, including any subsequence having a size of a single base up to a subsequence that is one base shorter than the nucleotide sequence.

The phrase “sequencing run” refers to any step or portion of a sequencing experiment performed to determine some information relating to at least one biomolecule (e.g., nucleic acid molecule).

As used herein, the phrase “dNTP” means deoxynucleotidetriphosphate, where the nucleotide comprises a nucleotide base, such as A, T, C, G or U.

The term “monomer” as used herein means any compound that can be incorporated into a growing molecular chain by a given polymerase. Such monomers include, without limitations, naturally occurring nucleotides (e.g., ATP, GTP, TTP, UTP, CTP, dATP, dGTP, dTTP, dUTP, dCTP, synthetic analogs), precursors for each nucleotide, non-naturally occurring nucleotides and their precursors or any other molecule that can be incorporated into a growing polymer chain by a given polymerase.

A “polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus (Taq) DNA polymerase, Thermus thermophilus (Tth) DNA polymerase, Vent DNA polymerase (New England Biolabs), Deep Vent DNA polymerase (New England Biolabs), Bacillus stearothermophilus (Bst) DNA polymerase, DNA Polymerase Large Fragment, Stoeffel Fragment, 9° N DNA Polymerase, 9° Nₘ polymerase, Pyrococcus furiosis (Pfu) DNA Polymerase, Thermus filiformis (Tfl) DNA Polymerase, RepliPHI Phi29 Polymerase, Thermococcus litoralis (Tli) DNA polymerase, eukaryotic DNA polymerase beta, telomerase, Therminator polymerase (New England Biolabs), KOD HiFi. DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting and/or molecular evolution, and polymerases cited in U.S. Pat. Appl. Pub. No. 2007/0048748 and in U.S. Pat. Nos. 6,329,178; 6,602,695; and 6,395,524. These polymerases include wild-type, mutant isoforms, and genetically engineered variants such as exo-polymerases; polymerases with minimized, undetectable, and/or decreased 3′→5′ proofreading exonuclease activity, and other mutants, e.g., that tolerate labeled nucleotides and incorporate them into a strand of nucleic acid. In some embodiments, the polymerase is designed for use, e.g., in real-time PCR, high fidelity PCR, next-generation DNA sequencing, fast PCR, hot start PCR, crude sample PCR, robust PCR, and/or molecular diagnostics. Such enzymes are available from many commercial suppliers, e.g., Kapa Enzymes, Finnzymes, Promega, Invitrogen, Life Technologies, Thermo Scientific, Qiagen, Roche, etc.

The term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, a “system” denotes a set of components, real or abstract, comprising a whole where each component interacts with or is related to at least one other component within the whole.

As used herein the term “isolating” is intended to mean that the material in question exists in a physical milieu distinct from that in which it occurs in nature and/or has been completely or partially separated, isolated, or purified from other components (e.g., other nucleic acid molecules).

As used herein, the term “solid phase carrier” is an entity that has, or to which can be added, a functional group (one or more) that reversibly binds the target species, e.g., to provide a “capture substrate”. The solid phase carrier is essentially insoluble under conditions in which a target species can be precipitated onto (can bind to) the solid phase carrier. Suitable solid phase carriers for use in the methods of the present technology have sufficient surface area to permit efficient binding of the target species to the functional group(s) on the carriers, and are further characterized by having surfaces which are capable of reversibly binding the target species. Suitable solid phase carriers include, but are not limited to, microparticles (e.g., beads), fibers, and supports that have an affinity for a target species, such as nucleic acid, and which can embody a variety of shapes, that are either regular or irregular in form, and preferably have a shape that maximizes the surface area of the solid phase, and embodies a carrier which is amenable to microscale manipulations. In one embodiment, the solid phase carrier is a magnetic microparticle (e.g., a paramagnetic (magnetically responsive) microparticle).

As used herein, “paramagnetic microparticles” refer to microparticles that respond to an external magnetic field (e.g., as produced by a rare earth (e.g., neodymium) magnet) but which demagnetize when the field is removed. Thus, the paramagnetic microparticles are efficiently separated from a solution using a magnet, but can be easily resuspended without magnetically induced aggregation occurring. Particular paramagnetic microparticles comprise a magnetite rich core encapsulated by a polymer shell. In one embodiment, suitable paramagnetic microparticles have a magnetite/encapsulation ratio of approximately 20-35%. For example, magnetic particles having a magnetite/encapsulation ratio of approximately 23%, 25%, 28%, 30%, 32%, or 34% are suitable for use in the present technology. Magnetic particles having less than approximately a 20% ratio are only weakly attracted to the magnets used to accomplish magnetic separations. Depending on the nature of the mixture used in the methods of the present technology, some embodiments comprise use of paramagnetic microparticles having a higher percentage of magnetite. The use of encapsulated paramagnetic microparticles, having no exposed iron, or Fe₃O₄, on their surfaces, eliminates or minimizes the possibility of iron interfering with certain downstream manipulations of the isolated nucleic acid (e.g., polymerase function).

### Aspects of the Technology

The technology described herein provides a NGS library workflow that requires fewer steps, less hands on time and turnaround time, a reduced number of tube transfers, pipet steps, and decreased cost compared to conventional technologies. The methods described in this disclosure are NGS platform agnostic and can be used with other nucleic acid analysis techniques involving sequencing or otherwise.

### Methods

Some embodiments provide methods for preparing NGS libraries ready for use in a NGS workflow. In general, method embodiments comprise capturing a defined (e.g., limited) quantity of a NGS library (e.g., less than 250 ng and/or less than 100 ng, e.g., to provide a concentration of less than 1 nM, e.g., less than 0.1 to 0.5 nM, less than 0.05 nM nucleic acid), e.g., using modified (e.g. carboxyl-modified) magnetic beads, after library fragments are generated from regions of interest in a nucleic acid (e.g., an RNA or DNA) and, in some embodiments, formatted with sequence platform-specific adapters. The method comprises use of an amount and type of a capture substrate having a known and defined binding capacity for capturing nucleic acids. The capture substrate is added to a library preparation (e.g., a fragment library or amplicon panel) that is known to have more nucleic acid than the biding capacity of the capture substrate. As such, the technology provides for the capture of a defined quantity of nucleic acids from the library (that is less than the total amount of nucleic acids present in the library), thus providing a normalized preparation, e.g., a sample having a known amount (e.g., within a known range and/or within a small known error window) of nucleic acids for use in NGS platforms.

In some embodiments of the methods, the methods comprise steps such as: providing a NGS library, formatting the NGS library for next generation sequencing (e.g., comprising attaching adapters), combining the formatted NGS library with a defined recovery-limiting type and/or amount of a capture substrate (e.g., carboxyl-modified magnetic beads), preferentially binding to the capture substrate library fragments of a desired size range (e.g., greater than 100 bases or bp, e.g., greater than 10 bases or by and less than 1000, 2000, 3000, 4000, or 5000 bases or bp) relative to library fragments outside the desired size range (e.g., by using buffer conditions (e.g., salt concentrations and/or pH) that promote binding of library fragments of the desired size range to the capture substrate and that do not promote binding of library fragments outside of the desired size range to the capture substrate), capturing bound library fragments (e.g., using a magnet), removing excess unbound library fragments, washing bound library fragments, eluting bound library fragments, collecting eluted library fragments, diluting eluted library fragments, and sequencing eluted library fragments.

For example, in preferred embodiments, the technology is related to a method for normalizing the concentration of a NGS library, the method consisting of, comprising, or consisting essentially of mixing a next-generation sequencing library comprising a first amount of library fragments with a capture substrate (e.g., comprising a paramagnetic microparticle functionalized with a carboxyl group) having a capacity to bind a second amount of library fragments that is less than the first amount of library fragments to provide a capture mixture comprising unbound library fragments and a capture substrate comprising bound library fragments. In some embodiments, a subsequent step comprises eluting the bound library fragments from the capture substrate to provide a concentration normalized NGS library. In some embodiments, the methods further comprise steps that occur after the mixing step and before the eluting step such as: removing the unbound library fragments from the capture mixture; washing the capture substrate comprising the bound library fragments; size-selecting the NGS library (e.g., by adjusting buffer components and/or adjusting ionic strength); and/or adding a nucleic acid precipitating reagent (e.g., PEG). In some embodiments, the methods comprise steps that occur after the eluting step such ligating an adapter to a library fragment and/or combining two or more concentration normalized NGS libraries to provide a multiplex concentration normalized NGS library.

In particular embodiments, the methods comprise mixing a next-generation sequencing library comprising a first amount of library fragments with a capture substrate (e.g., comprising a paramagnetic microparticle functionalized with a carboxyl group) having a capacity to bind a second amount of library fragments that is less than the first amount of library fragments to provide a capture mixture comprising unbound library fragments and a capture substrate comprising bound library fragments, wherein the ratio of the first amount of library fragments to the second amount of library fragments is more than 1000, more than 100, or more than 10; wherein the concentration normalized NGS library comprises DNA at a concentration of less than 1 nM, less than 0.75 nM, less than 0.55 nM, or less than 0.25 nM; wherein the concentration normalized NGS library comprises DNAs that comprise more than 100 bp; wherein the concentration normalized NGS library comprises less than 200, less than 150, less than 100, less than 50, less than 25, less than 10, or less than 5 library fragments; wherein the next-generation sequencing library comprises less than 250 ng, less than 200, less than 150, or less than 100 ng of DNA; and/or wherein the steps of the method are performed in a single vessel.

In some embodiments, the geometry of the capture substrate (e.g., surface-modified magnetic beads), the size of the particles comprising the capture substrate, and buffer components are selected to provide the desired normalization, library purification, and size selection specifically for NGS library production. For example, surface-modified magnetic bead size can be increased or decreased in the formulation to alter the available surface area to achieve a desired concentration normalization. The capacity of the bead for binding a nucleic acid scales with the surface area of the bead. Thus, as the diameter of the bead increases, the surface area increases and the capacity for binding a nucleic acid increases. In addition, the roughness of the bead is related to the surface area such that a bead having a rough or undulated surface has a greater surface area and a greater capacity for binding nucleic acids than a smooth bead having the same diameter. In addition, the capacity of the bead scales with the density of nucleic acid binding groups per unit of surface area. Thus, as the number of nucleic acid binding groups per unit of surface area increases, the binding capacity of a bead increases. The binding capacity of beads selected for use in the technology can be determined empirically, e.g., by quantifying the binding of a series of standards comprising known amounts of nucleic acids.

The size, type of surface modification, concentration, and buffer components are varied in embodiments of the technology as appropriate for a fragment library based on the expected fragment size range and the expected library fragment yield range for the type of NGS library produced and used as input to the method.

In some embodiments, the capture substrate comprises a paramagnetic microparticle (e.g., a “magnetic bead”). In embodiments comprising use of paramagnetic microparticles, the paramagnetic microparticles are preferably separated from solutions using magnetic means, such as applying a magnetic field of at least 1000 Gauss. However, other methods known to those skilled in the art can be used to remove the magnetic microparticles from the supernatant (e.g., vacuum filtration or centrifugation). The remaining solution can then be removed, leaving solid phase carriers having the nucleic acid of the cell adsorbed to their surface.

In some embodiments, the methods produce a NGS library that is immediately ready for loading onto the NGS sequencer workflow without further dilution. In embodiments of methods where no additional dilution is required, multiplex sequencing of multiple samples simply requires the combination of equal volumes from each final, concentration normalized, NGS library sample produced by the methods prior to sequencer workflow loading. In some embodiments, the disclosed methods produce an NGS library whose concentration has been normalized and from which a sample ready for NGS workflow is produced by a single dilution prior to loading onto the NGS sequencer workflow.

In some embodiments, the methods provide a NGS library comprising a concentration of DNA less than 1 nM, e.g., less than 0.90 nM, less than 0.80 nM, less than 0.70 nM, less than 0.60 nM, less than 0.55 nM, less than 0.50 nM, less than 0.45 nM, less than 0.40 nM, less than 0.35 nM, less than 0.30 nM, less than 0.25 nM, less than 0.20 nM, less than 0.15 nM, or less than 0.10 nM. The input NGS library that is used for input into embodiments of the technology comprises less than 100 nM, less than 90 nM, less than 80 nM, less than 70 nM, less than 60 nM, less than 50 nM, less than 40 nM, less than 30 nM, less than 20 nM, less than 25 nM, less than 20 nM, less than 15 nM, less than 10 nM, less than 9 nM, less than 8 nM, less than 7.5 nM, less than 7 nM, less than 6.5 nM, less than 6 nM, less than 5.5 nM, or less than 5 nM of DNA. In some embodiments, the input DNA to be normalized with the technology comprises a mass less than 250 ng, less than 200 ng, less than 150 ng, less than 100 ng, less than 75 ng, less than 50 ng, or less than 25 ng of DNA. For example, in some embodiments of the technology, limited amplification is performed prior to normalization to retain coverage uniformity across amplicons present within an amplicon panel.

In some embodiments, the technology provides a NGS library comprising a relatively low number of nucleic acids (e.g., fragments or amplicons), e.g., comprising less than 500 nucleic acids, less than 450 nucleic acids, less than 400 nucleic acids, less than 350 nucleic acids, less than 300 nucleic acids, less than 250 nucleic acids, less than 200 nucleic acids, less than 150 nucleic acids, less than 100 nucleic acids, less than 50 nucleic acids, less than 25 nucleic acids, e.g., 1 to 150 nucleic acids.

In some embodiments, the methods and formulations are used for concentration normalization, purification, and size selection accomplished in a single step and/or in a single vessel (e.g., a single tube, well, or other sample-holding object).

In some embodiments, the technology provides a NGS library comprising fragments having a length greater than 75 bp or bases, greater than 80 bp or bases, greater than 85 bp or bases, greater than 90 bp or bases, greater than 95 bp or bases, greater than 100 bp or bases, greater than 105 bp or bases, greater than 110 bp or bases, greater than 115 bp or bases, greater than 120 bp or bases, e.g., in some embodiments, fragments of approximately 100 bp or shorter are efficiently removed during concentration normalization.

### Capture Substrates

In some embodiments, the technology comprises binding a nucleic acid (e.g., a NGS library) to a capture substrate. In some embodiments, capture is non-specific, e.g., the capture substrate does not have specificity for nucleic acids of a particular size and/or composition, but binds to all nucleic acids with substantially equivalent affinity. In some embodiments, the capture substrate has a relatively higher affinity for a particular type or class of nucleic acids than for another type or class of nucleic acids. For example, in some embodiments, the capture substrate is specific for nucleic acids greater than 1000 bp long but not nucleic acids less than 1000 bp long. In some embodiments, the capture substrate is specific for nucleic acids having a particular composition (e.g., having a poly-A tail, high or low GC content, etc.), structure (stem-loop, linear, circular, etc.), modification (e.g., methylated or not methylated), and/or sequence.

In some embodiments, the capture substrate and/or a composition comprising a capture substrate has a capacity for binding nucleic acids that is less than 250 ng or less than 100 ng (e.g., less than 250 ng, 200 ng, 150 ng, 100 ng, 75 ng, 50 ng, 25 ng, or 10 ng or less).

In some embodiments, amplicons of a NGS library are bound to a capture substrate that binds nucleic acids, e.g., the capture substrate comprises free COOH or COO— (carboxyl) groups. In some embodiments, the capture substrate comprises a magnetic particle (e.g., a paramagnetic particle).

In some embodiments, suitable paramagnetic microparticles have a size that is large enough to provide for their separation from solution, for example by a magnetic field or by filtration. In some embodiments, the paramagnetic microparticles have a size that is large enough to provide an appropriate surface area and volume for microscale manipulation. For example, in some embodiments, sizes range from approximately 0.1 μm mean diameter to approximately 100 μm mean diameter, e.g., approximately 1.0 μm mean diameter. Suitable magnetic microparticles for use in the present technology are available from commercial suppliers such as Agencourt Biosciences, Polysciences, Bioclone, Seradyne, and Bangs Laboratories Inc., Fishers, Ind. (e.g., Estapor® carboxylate-modified encapsulated magnetic microspheres).

In some embodiments, amplicons of a NGS library bind non-specifically to at least one functional group on the solid phase carrier. “Non-specific binding” refers to binding of different target species molecules (e.g., different species of nucleic acid, such as nucleic acids that differ in size) with approximately similar affinity to the functional groups on the solid phase carriers, despite differences in the structure (e.g., nucleic acid sequence) or size of the different target species molecules. The binding can occur, for example, via facilitated adsorption. As used herein, “facilitated adsorption” refers to a process whereby a crowding reagent (e.g., PVP) or a precipitating reagent (e.g., a poly-ethylene glycol, ethanol, isopropanol) is used to promote the precipitation and subsequent adsorption of a species of DNA molecules, which were initially in mixture, onto the surface of a solid phase carrier (capture substrate).

In some embodiments, nucleic acids (e.g., fragments or amplicons) of a NGS library bind specifically (selectively) to at least one functional group on the solid phase carrier. “Specific binding” or “selective binding” refers to binding of, for example, particular nucleic acid molecules (e.g., a target nucleic acid species) to one or more functional groups on the solid phase carriers to the exclusion of other nucleic acid species in a mixture. In this embodiment, the functional group has a greater affinity for particular nucleic acid molecules (e.g., the target nucleic acid species) than other nucleic acid molecules.

The solid phase carriers used in the methods of the present technology have a functional group coated surface. As used herein, the term “functional group-coated surface” refers to a surface of a solid phase carrier that is coated with functional groups or moieties that reversibly bind one or more nucleic acids of a NGS library, either directly (the functional group binds the nucleic acid) or indirectly (the functional group binds a group that is linked to the nucleic acid).

Methods for coating solid phase carriers with functional groups, either directly or indirectly, are known in the art. For example, embodiments are provided in which the functional groups (e.g., COOH/COO—) coat a solid phase carrier during formation of the solid phase carrier. See, for example, U.S. Pat. No. 5,648,124, which is incorporated herein by reference. In addition, embodiments are provided in which solid phase carriers are coated with functional groups by covalently coupling a functional group (one or more) to a COOH group (one or more) on the solid phase carrier. A particular example of a functional group coated surface is a surface that is coated with moieties that each has a free functional group that is bound to the amino group of the amino silane of the microparticle; as a result, the surfaces of the microparticles are coated with the functional group containing moieties. The functional group acts as a bioaffinity adsorbent for precipitated nucleic acid (e.g., polyalkylene glycol precipitated DNA).

In some embodiments, capture substrates comprise a functional group that is a carboxylic acid (COOH/COO—). A suitable moiety with a free carboxylic acid functional group is a succinic acid moiety in which one of the carboxylic acid groups is bonded to the amine of an amino silane through an amide bond and the second carboxylic acid is unbonded, resulting in a free carboxylic acid group attached or tethered to the surface of the solid phase carrier. Carboxylic acid-coated magnetic particles are commercially available from, for example, Polysciences, Inc. Carboxy groups provide for the effective elution of nucleic acid from a solid phase carrier. Carboxy groups have a pKa of approximately 4.7 and are thus negatively charged at neutral pH. Nucleic acid, such as DNA, is negatively charged; thus, in the absence of a crowding reagent or salt, nucleic acid is repelled from a carboxy-modified microparticle at neutral pH.

Embodiments provide solid phase carriers having a functional group coated surface that reversibly binds nucleic acid molecules, e.g., to provide a capture substrate. Exemplary capture substrates include, but are not limited to, magnetically responsive solid phase carriers having a functional group-coated surface, such as, but not limited to, amino-coated, carboxyl-coated, and encapsulated carboxyl group-coated paramagnetic microparticles.

In some embodiments, other functional groups are coupled to the solid phase carriers through carboxydiimide coupling to carboxy groups on the surface of the solid phase carrier. Solid phase carriers having a high density of carboxyl groups on the surface can be contacted with another functional group (e.g., oligo-dT) that binds to some but not all of the carboxy groups through carbodiimide coupling. Sufficient carboxy functional groups remain (which can be used, for example, to bind nucleic acid) following carbodiimide coupling to a distinct functional group resulting in a solid phase carrier having dual functionality wherein binding of nucleic acid to the carboxy groups and a binding of a separate moiety to the second functional group can occur. Thus, the solid phase carriers can be used to remove or retain another target molecule.

Functional groups that bind target species, such as nucleic acids and peptides, are well known in the art (e.g., see Hermanson, G. T., Bioconjugate Techniques, Academic Press, San Diego, Calif. (1996), which is incorporated herein by reference). Functional groups that bind nucleic acids directly include, for example, metal ions, an amine group, a carboxyl group, an encapsulated carboxyl group, silica (SiOH), diethyl aminoethyl (DEAE), and a group that hybridizes to a nucleic acid sequence in the mixture.

A functional group that hybridizes to a nucleic acid sequence can be a nucleic acid sequence that is complementary to all or a portion of a nucleic acid in the mixture (e.g., complementary to all or a portion of the nucleic acid sequence of the target nucleic acid sequence to be isolated). For example, in some embodiments, the nucleic acid sequence that is complementary is a sequence that is specific to (characteristic of) the nucleic acid species to be isolated so that substantially all the nucleic acid (the majority of nucleic acid species) in the mixture that bind the complementary sequence comprise the target nucleic acid species, while other nucleic acid sequences present in the mixture do not bind to the complementary sequence. For example, the group can be an oligodeoxythymidine (oligo dT) group which is a polymer of deoxyribothymidine and is complementary to the adenine nucleotide polymer (polyadenylate (poly A) tail) at the 3′ end of messenger RNA (mRNA), and is a sequence that is characteristic of mRNA or a cDNA made from an mRNA. Oligo dT groups can be a polymer of from approximately 3 to approximately 100 thymidines, from approximately 5 to approximately 75 thymidines, from approximately 8 to approximately 60 thymidines, from approximately 10 to approximately 50 thymidines, from approximately 15 to approximately 40 thymidines, or from approximately 20 to approximately 30 thymidines. Modified oligo dT groups can also be used in the methods of the present technology. For example, an oligo dT wherein the last two 3′ nucleotides are N or an oligo dT, wherein the last two 3′ nucleotides are VN, where “N” is adenine (A), cytosine (C), thymidine (T), or guanidine (G), and “V” is A, C, or G can be used.

Groups that bind target nucleic acid indirectly bind to a moiety—such as a label or tag—that is attached to the nucleic acid. Therefore, nucleic acid comprising a tag that can bind to a functional group on the solid phase carrier can be isolated using the methods of the present technology. Such groups include, for example, groups that interact with a binding partner. For example, the functional groups can be a binding partner that is conventionally used to isolate particular biomolecules based on their composition or sequence. Examples of such functional groups for use in the methods of the present technology include avidin, streptavidin, biotin, an antibody, an antigen, a sequence-specific interaction (a hybridizable tag), DNA specific binding protein (e.g., finger domains, transcription factors), and derivatives thereof.

In a particular embodiment, the functional group is biotin or a molecule that comprises biotin. Biotin, a water-soluble vitamin, is used extensively in biochemistry and molecular biology for a variety of purposes including macromolecular detection, purification, and isolation, and in cytochemical staining (see, e.g., U.S. Pat. No. 5,948,624, which is incorporated herein by reference). The utility of biotin arises from its ability to bind strongly to the tetrameric protein avidin, found in egg white and the tissues of birds, reptiles and amphibians, or to its chemical cousin, streptavidin, which is slightly more specific for biotin than is avidin. The biotin interaction with avidin is among the strongest non-covalent affinities known, exhibiting a dissociation constant of approximately 1.3×10⁻¹⁵ M (Hermanson, G. T., Bioconjugate Techniques, Academic Press, San Diego, Calif. (1996), p. 570). In other embodiments, the functional group is biocytin and/or a biotin analog (e.g., biotin amido caproate-hydroxysuccinimide ester, biotin-PEO4-N-hydroxysuccinimide ester, biotin 4-amidobenzoic acid, biotinamide caproyl hydrazide) and biotin derivatives (e.g., biotin-dextran, biotin-disulfide-N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides, biotinylated amino acids such as Nε-biotinyl-1-lysine) (see, e.g., U.S. Pat. No. 5,948,624).

In another embodiment, the functional group is avidin or is a molecule that comprises avidin (avidinylated). Avidin is a glycoprotein found in egg whites that contains four identical subunits, each of which possesses a binding site for biotin (Hermanson, G. T., Bioconjugate Techniques, Academic Press, San Diego, Calif. (1996), p. 570). Streptavidin and other avidin analogs can also be used in the methods of the present technology. Such avidin analogs include, e.g., avidin conjugates, streptavidin conjugates, highly purified and/or fractionated species of avidin or streptavidin, non- or partial amino acid variants of avidin or streptavidin (e.g., recombinant or chemically synthesized avidin analogs with amino acid or chemical substitutions which still allow for high affinity, multivalent, or univalent binding of the avidin analog to biotin). Streptavidin is another biotin-binding protein that is isolated from Streptomyces avidinii (Hermanson, supra).

The functional group can also be an antibody. As used herein, the term “antibody” encompasses both polyclonal and monoclonal antibodies (e.g., IgG, IgM, IgA, IgD, and IgE antibodies). The terms polyclonal and monoclonal refer to the degree of homogeneity of an antibody preparation, and are not intended to be limited to particular methods of production. Any antibody or antigen-binding fragment can be used in the methods of the technology. For example, single chain antibodies, chimeric antibodies, mammalian (e.g., human) antibodies, humanized antibodies, CDR-grafted antibodies (e.g., primatized antibodies), veneered antibodies, multivalent antibodies (e.g., bivalent), and bispecific antibodies are encompassed by the present technology and the term “antibody”. Chimeric, CDR-grafted, or veneered single chain antibodies, comprising portions derived from different species, are also encompassed by the present technology and the term “antibody”. The various portions of these antibodies can be joined together chemically by conventional techniques or can be prepared as a contiguous protein using genetic engineering techniques. For example, nucleic acids encoding a chimeric or humanized chain can be expressed to produce a contiguous protein. See, e.g., U.S. Pat. No. 4,816,567; European Patent No. 0,125,023 B1; U.S. Pat. No. 4,816,397; European Patent No. 0,120,694 B1; WO 86/01533; European Patent No. 0,194,276 B1; U.S. Pat. No. 5,225,539; European Patent No. 0,239,400 B1; European Patent No. 0 451 216 B1; EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Alternatively, the functional group can be an antigen. As used herein, the term “antigen”, “immunogen”, or “epitope” (e.g., T cell epitope, B cell epitope) refers to a substance for which an antibody or antigen-binding fragment has binding specificity. The antibodies and antigen-binding fragments for use in the methods of the technology have binding specificity for a variety of immunogens (e.g., polypeptides).

In some embodiments, the capture substrate comprises one or more heterologous functional groups. Any number of heterologous (distinct) functional groups (e.g., heterobifunctional, heterotrifunctional, heteromultifunctional) can be present on the surface of the solid phase particles as long as the presence of the functional groups do not interfere (e.g., chemically, sterically) with the reversible binding of nucleic acids. In one embodiment, there is a functional group from approximately every 2 A² up to approximately 200 A².

The capacity of a solid substrate such as a bead can be determined using a variety of techniques. In some embodiments, the capacity of a solid substrate such as a bead is determined empirically, e.g., using a defined solid substrate, a set of standard samples comprising known amounts of nucleic acids, and testing the capacity of the solid substrate under defined conditions.

In some embodiments, the capacity of a solid substrate such as a bead is estimated, determined, or predicted using the known characteristics of the bead. For example, embodiments comprise use of several different strategies for binding, selection, purification, and concentration normalization of nucleic acids (e.g., an NGS library), e.g., COOH/SPRI, oligo hybridization, biotin/streptavidin. In preferred embodiments described herein, COOH modified beads are used in a solid phase reversible immobilization (SPRI) method. Further, in some embodiments, a “crowding agent” (e.g., PEG) and a salt are used to drive negatively charged DNA to associate/precipitate with carboxyl groups on the bead surface (see, e.g., DeAngelis, et al (1995) “Solid-phase reversible immobilization for the isolation of PCR products” Nucleic Acids Res. 23(22):4742-3). In some embodiments, the DNA fragment sizes that bind to the COOH beads are determined by the concentration of PEG and salt. In particular, the higher the concentration of PEG and salt, the smaller the size cut-off of the DNA that binds to the beads.

In addition, several exemplary characteristics of a solid support (e.g., a bead) are used to predict capacity. For instance, assuming a bead is a smooth sphere, some exemplary characteristics and relationships for predicting bead capacity include: bead radius (e.g., in nm), total available surface area (e.g., 4πr²), mass of one bead (e.g., g), functional group density per bead (e.g., number of functional groups/nm²), and number of functional groups that associate per DNA fragment binding event.

Further, the binding capacity for DNA can be determined, estimated, and/or predicted as follows:

DNA binding capacity=[Total available surface area]×[Functional group density]×[Number of functional groups consumed per DNA fragment binding event].

An exemplary calculation provides an estimate of DNA binding capacity. Assuming the surface comprises one COOH group per nm² of surface area and that 10 COOH groups are consumed per DNA fragment binding event, an estimate of bead binding capacity includes the following calculations:

For a 1-μm bead, the bead capacity estimate is [3,141,500 nm²]×[1 COOH group/nm²]×[1 DNA frag/10 COOH groups]=314,150 DNA fragments/bead.

For an 8-μm bead, the bead capacity estimate is [201,056,000 nm²]×[1 COOH group/nm²]×[1 DNA frag/10 COOH groups]=20,105,600 DNA fragments/bead.

These values are 31,415 and 2,010,560, respectively for an assumption that 1 COOH groups are consumed per DNA fragment binding event. Thus, for the range of 1 to 10 COOH groups consumed per DNA fragment binding event, the capacity is predicted to range from approximately 30,000 to 300,000 DNA fragments per 1-μm bead; the capacity is predicted to range from approximately 2,000,000 to 20,000,000 DNA fragments per 8-μm bead. Accordingly, holding the total mass of beads in the reaction constant (e.g., 0.1% solids=1 mg beads/1 mL reaction), then the total DNA binding capacity per reaction is significantly greater when using a smaller bead size. That is, the 1-μm beads have a higher surface area per unit mass compared to the 8-μm beads. See Table 1.

TABLE 1
estimated DNA binding capacities of 1-μm and 8-μm beads

| Bead | | Reaction | Bead | | | DNA binding | DNA binding |
|---|---|---|---|---|---|---|---|
| Size | Single Bead | Volume | Concentration | # total beads | Total bead surface area | capacity per | capacity per |
| (μm) | Mass (g) | (uL) | (% solids) | per reaction | per reaction (nm²) | bead | reaction |
| 1 | 7.9E−13 | 75 | 0.1 | 94,861,060 | 3.0E+14 | 314,150 | 3.0.E+13 |
| 8 | 4.0E−10 | 75 | 0.1 | 185,277 | 3.7E+13 | 20,105,600 | 3.7.E+12 |

### Buffers

In the methods of the present technology, the mixture comprising the NGS library and the solid phase carriers is maintained under conditions appropriate for binding of the nucleic acids of the NGS library to the functional groups on the carriers. In some embodiments, the methods and agents (reagents) described herein are used together with a variety of purification techniques (e.g., nucleic acid purification techniques) that involve binding of nucleic acid to solid phase carriers, including those described in, e.g., U.S. Pat. Nos. 5,705,628; 5,898,071; 6,534,262; WO 99/58664; U.S. Pat. Appl. Pub. No. 2002/0094519 A1, U.S. Pat. Nos. 5,047,513; 6,623,655; and 5,284,933, the contents of which are herein incorporated by reference.

As described herein, one or more agents (e.g., buffers, enzymes) is/are used to bind or remove the nucleic acids (e.g., amplicons or library fragments) from the solid phase carriers. In various embodiments, the components of the agents that promote association (e.g., binding) and/or disassociation of the target nucleic acids with the solid phase carriers (capture substrate) are present in one agent or in multiple agents (e.g., a first agent, a second agent, a third agent, etc.). Accordingly, when more than one agent is used in the methods of the present technology, embodiments provide that the agents are used simultaneously or sequentially. Depending on the purpose for which the methods described herein are used, one of skill in the art can determine the number and order of agents to be used in the methods of the present technology.

In some embodiments, the agent is used in the methods of the present technology to cause the nucleic acids (e.g., library fragments or amplicons of the NGS library) in the mixture to precipitate or adsorb onto the functional groups on the surface of the solid phase carriers (a nucleic acid precipitating agent). In one embodiment, a nucleic acid precipitating agent is used at a sufficient concentration to precipitate the nucleic acid of the mixture onto the solid phase carrier.

A “nucleic acid precipitating reagent” or “nucleic acid precipitating agent” is a composition that causes a nucleic acid to go out of solution. Suitable precipitating agents include alcohols (e.g., short chain alcohols, such as ethanol or isopropanol) and poly-OH compounds (e.g., a polyalkylene glycol). The nucleic acid precipitating reagent can comprise one or more of these agents. The nucleic acid precipitating reagent is present in sufficient concentration to bind the nucleic acid onto the solid phase carriers nonspecifically and reversibly. Such nucleic acid precipitating agents can be used, for example, to bind nucleic acids non-specifically, or nucleic acids specifically, depending on the concentrations used, to solid phase carriers, e.g., solid phase carriers comprising COOH as a functional group.

In one embodiment, carboxy-based magnetic beads are used that involve binding nucleic acids to carboxyl coated solid phase carriers (e.g., magnetic and/or paramagnetic microparticles) using various nucleic acid precipitating reagents or crowding reagents such as alcohols, glycols (e.g., alkylene, polyalkylene glycol, ethylene, polyethylene glycol), and polyvinyl pyrrolidinone (PVP) (e.g., polyvinyl pyrrolidinone-40). In some embodiments, the molecular weights of these precipitating and/or crowding reagents are adjusted to produce low viscosity solutions with substantial precipitating power. In some embodiments, size-specific nucleic acid isolation is performed by either adjusting the concentration of the precipitating and/or crowding reagents, the molecular weight of the precipitating and/or crowding reagents, or by adjusting the salt, pH, polarity, or hydrophobicity of the solution. Large nucleic acid molecules are precipitated and/or crowded out of solution at low concentrations of salt, precipitating, and/or crowding reagents, whereas the smaller nucleic acid molecules are precipitated and/or adsorbed at higher concentrations of precipitating and/or crowding reagents. See, for example, U.S. Pat. No. 5,705,628; U.S. Pat. No. 5,898,071; U.S. Pat. No. 6,534,262 and U.S. Published Application No. 2002/0106686, all of which are incorporated herein by reference.

Appropriate alcohol (e.g., ethanol, isopropanol) concentrations (final concentrations) for use in the methods of the present technology are from approximately 5% to approximately 100%; from approximately 40% to approximately 60%; from approximately 45% to approximately 55%; and from approximately 50% to approximately 54%, described as a volume:volume ratio.

Appropriate polyalkylene glycols include polyethylene glycol (PEG) and polypropylene glycol. Suitable PEG can be obtained from Sigma (Sigma Chemical Co., St. Louis Mo., Molecular weight 8000, Dnase and Rnase free, Catalog number 25322-68-3). The molecular weight of the polyethylene glycol (PEG) can range from approximately 250 to approximately 10,000; from approximately 1000 to approximately 10,000; from approximately 2500 to approximately 10,000; from approximately 6000 to approximately 10,000; from approximately 6000 to approximately 8000; from approximately 7000 to approximately 9000; from approximately 8000 to approximately 10,000. In general, the presence of PEG provides a hydrophobic solution that forces hydrophilic nucleic acid molecules out of solution. In one embodiment, the PEG concentration is from approximately 5% to approximately 20%. In other embodiments, the PEG concentration ranges from approximately 7% to approximately 18%; from approximately 9% to approximately 16%; and from approximately 10% to approximately 15%, described as a weight:volume ratio.

Optionally, salt may be added to the reagent to cause precipitation of the nucleic acid in the mixture onto the solid phase carriers. Suitable salts that are useful for facilitating the adsorption of nucleic acid molecules targeted for isolation to the magnetically responsive microparticles include sodium chloride (NaCl), lithium chloride (LiCl), barium chloride (BaCl₂), potassium chloride (KCl), calcium chloride (CaCl₂), magnesium chloride (MgCl₂), and cesium chloride (CsCl). In some embodiments, sodium chloride is used. In general, the salt minimizes the negative charge repulsion of the nucleic acid molecules. The wide range of salts suitable for use in the method indicates that many other salts can also be used and suitable levels can be empirically determined by one of ordinary skill in the art. The salt concentration can be from approximately 0.005 M to approximately 5 M, from approximately 0.1 M to approximately 0.5 M; from approximately 0.15 M to approximately 0.4 M; and from approximately 2 M to approximately 4 M.

In embodiments in which the functional group is a sequence that is complementary, and thus hybridizes, to one or more nucleic acids in the mixture, a hybridizing buffer can be used for binding. Suitable buffers for use in such a method are known to those of skill in the art. An example of a suitable buffer is a buffer comprising NaCl (e.g., approximately 0.1 M to approximately 0.5 M), Tris-HCl (e.g., 10 mM), EDTA (e.g., 0.5 mM), sodium citrate (SSC), and combinations thereof.

A suitable “elution buffer” for use in the methods of the present technology is a buffer that elutes (e.g., selectively) target nucleic acid from the functional group(s) of the solid phase carriers. In some embodiments, the elution buffer is water or an aqueous solution. For example, useful buffers include, but are not limited to, Tris-HCl (e.g., 10 mM, pH 7.5), Tris acetate, sucrose (20% w/v), EDTA, and formamide (e.g., at 90% to 100%) solutions. In some embodiments, the elution buffer is a buffered salt solution comprising a monovalent (one or more) cation such as sodium, lithium, potassium, and/or ammonium (e.g., from approximately 0.1 M to approximately 0.5 M). Elution of nucleic acid from the solid phase carrier can occur quickly (e.g., in thirty seconds or less) when a suitable low ionic strength elution buffer is used.

In addition, impurities (e.g., proteins (e.g., enzymes), metabolites, chemicals, unincorporated nucleotides and/or primers, or cellular debris) can be removed from the solid phase carriers by washing the solid phase carriers with nucleic acid bound thereto (e.g., by contacting the solid phase carriers with a suitable wash buffer solution) before separating the solid phase carrier-bound target species from the solid phase carriers. As used herein, a “wash buffer” is a composition that dissolves or removes impurities that may be bound to a microparticle, associated with the adsorbed nucleic acid, or present in the bulk solution, but that does not solubilize the target nucleic acids absorbed onto the solid phase. The pH, solute composition, and concentration of the wash buffer can be varied according to the types of impurities that are expected to be present. For example, ethanol (e.g., 70% (v/v)) exemplifies a preferred wash buffer useful to remove excess PEG and salt. In one embodiment, the wash buffer comprises NaCl (e.g., 0.1 M), Tris (e.g., 10 mM), and EDTA (e.g., 0.5 mM). The solid phase carriers with bound nucleic acid can also be washed with more than one wash buffer solution. The solid phase carriers can be washed as often as required (e.g., one, two, three or more, e.g., three to five times) to remove the desired impurities. However, the number of washings is preferably limited to minimize loss of yield of the bound target species.

A suitable wash buffer solution has several characteristics. First, the wash buffer solution must have a sufficiently high salt concentration (a sufficiently high ionic strength) that the nucleic acid bound to the solid phase carriers does not elute from the solid phase carriers, but remains bound to the microparticles. A suitable salt concentration is greater than approximately 0.1 M and is preferably approximately 0.5 M. Second, the buffer solution is chosen so that impurities that are bound to the nucleic acid or microparticles are dissolved. The pH, solute composition, and concentration of the buffer solution can be varied according to the types of impurities that are expected to be present. Suitable wash solutions include the following: 0.5×saline-sodium citrate (SSC; A 20×stock solution comprises 3 M sodium chloride and 300 mM trisodium citrate (adjusted to pH 7.0 with HCl)); 100 mM ammonium sulfate, 400 mM Tris pH 9, 25 mM MgCl₂, and 1% bovine serum albumin (BSA); 1-4 M guanidine hydrochloride (e.g., 1 M guanidine HCl with 40% isopropanol and 1% Triton X-100); and 0.5 M NaCl. In one embodiment, the wash buffer solution comprises 25 mM Tris acetate (pH 7.8), 100 mM potassium acetate (KOAc), 10 mM magnesium acetate (Mg₂OAc), and 1 mM dithiothreitol (DTT; Cleland's Reagent). In another embodiment, the wash solution comprises 2% SDS, 10% Tween, and/or 10% Triton.

The components of the agents used in the methods of the present technology can be contained in a single agent (reagent) or as separate components. In embodiments in which separate components of the agent(s) are used, the components may be combined simultaneously or sequentially with the mixture. Depending on the particular embodiment, the order in which the elements of the combination are combined may not necessarily be critical. The nature and quantity of the components contained in the reagent are as described in the methods above. The reagent may be formulated in a concentrated form, such that dilution is desirable to obtain the functions and/or concentrations described in the methods herein.

### Adapters

Methods of the technology involve attaching an adapter to a nucleic acid (e.g., a nucleic acid (e.g., a library fragment of a NGS library or an amplicon of an amplicon library). In certain embodiments, the adapters are attached to a nucleic acid with an enzyme. The enzyme may be a ligase or a polymerase. The ligase may be any enzyme capable of ligating an oligonucleotide (single stranded RNA, double stranded RNA, single stranded DNA, or double stranded DNA) to another nucleic acid molecule. Suitable ligases include T4 DNA ligase and T4 RNA ligase (such ligases are available commercially, e.g., from New England Biolabs). Methods for using ligases are well known in the art. The ligation may be blunt-ended or via use of complementary over hanging ends. In certain embodiments, the ends of nucleic acids may be phosphorylated (e.g., using T4 polynucleotide kinase), repaired, trimmed (e.g. using an exonuclease), or filled (e.g., using a polymerase and dNTPs), to form blunt ends. Upon generating blunt ends, the ends may be treated with a polymerase and dATP to form a template independent addition to the 3′ end of the fragments, thus producing a single A overhanging. This single A is used to guide ligation of fragments with a single T overhanging from the 5′ end in a method referred to as T-A cloning. The polymerase may be any enzyme capable of adding nucleotides to the 3′ and the 5′ terminus of template nucleic acid molecules.

In some embodiments, the adapters comprise a universal sequence and/or an index, e.g., a barcode nucleotide sequence. Additionally, adapters can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters (e.g., a universal sequence), one or more restriction enzyme recognition sites, one or more overhangs complementary to one or more target polynucleotide overhangs, one or more probe binding sites (e.g. for attachment to a sequencing platform, such as a flow cell for massive parallel sequencing, such as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. Two or more sequence elements can be non-adjacent to one another (e.g. separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adapter oligonucleotide. When an adapter oligonucleotide is capable of forming secondary structure, such as a hairpin, sequence elements can be located partially or completely outside the secondary structure, partially or completely inside the secondary structure, or in between sequences participating in the secondary structure. For example, when an adapter oligonucleotide comprises a hairpin structure, sequence elements can be located partially or completely inside or outside the hybridizable sequences (the “stem”), including in the sequence between the hybridizable sequences (the “loop”). In some embodiments, the first adapter oligonucleotides in a plurality of first adapter oligonucleotides having different barcode sequences comprise a sequence element common among all first adapter oligonucleotides in the plurality. In some embodiments, all second adapter oligonucleotides comprise a sequence element common among all second adapter oligonucleotides that is different from the common sequence element shared by the first adapter oligonucleotides. A difference in sequence elements can be any such that at least a portion of different adapters do not completely align, for example, due to changes in sequence length, deletion or insertion of one or more nucleotides, or a change in the nucleotide composition at one or more nucleotide positions (such as a base change or base modification).

In some embodiments, an adapter oligonucleotide comprises a 5′ overhang, a 3′ overhang, or both that is complementary to one or more target polynucleotides. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs may comprise a random sequence of one or more nucleotides, such that one or more nucleotides are selected at random from a set of two or more different nucleotides at one or more positions, with each of the different nucleotides selected at one or more positions represented in a pool of adapters with complementary overhangs comprising the random sequence. In some embodiments, an adapter overhang is complementary to a target polynucleotide overhang produced by restriction endonuclease digestion. In some embodiments, an adapter overhang consists of an adenine or a thymine.

In some embodiments, the adapter sequences can contain a molecular binding site identification element to facilitate identification and isolation of the target nucleic acid for downstream applications. Molecular binding as an affinity mechanism allows for the interaction between two molecules to result in a stable association complex. Molecules that can participate in molecular binding reactions include proteins, nucleic acids, carbohydrates, lipids, and small organic molecules such as ligands, peptides, or drugs.

When a nucleic acid molecular binding site is used as part of the adapter, it can be used to employ selective hybridization to isolate a target sequence. Selective hybridization may restrict substantial hybridization to target nucleic acids containing the adapter with the molecular binding site and capture nucleic acids that are sufficiently complementary to the molecular binding site. Thus, through “selective hybridization” one can detect the presence of the target polynucleotide in an un-pure sample containing a pool of many nucleic acids. An example of a nucleotide-nucleotide selective hybridization isolation system comprises a system with several capture nucleotides that comprise complementary sequences to the molecular binding identification elements and are optionally immobilized to a solid support. In other embodiments, the capture polynucleotides could be complementary to the target sequences itself or a barcode or unique tag contained within the adapter. The capture polynucleotides can be immobilized to various solid supports, such as inside of a well of a plate, mono-dispersed spheres, microarrays, or any other suitable support surface known in the art. The hybridized complementary adapter polynucleotides attached on the solid support can be isolated by washing away the undesirable non-binding nucleic acids, leaving the desirable target polynucleotides behind. If complementary adapter molecules are fixed to paramagnetic spheres or similar bead technology for isolation, then spheres can be mixed in a tube together with the target polynucleotide containing the adapters. When the adapter sequences have been hybridized with the complementary sequences fixed to the spheres, undesirable molecules can be washed away while spheres are kept in the tube with a magnet or similar agent. The desired target molecules can be subsequently released by increasing the temperature, changing the pH, or by using any other suitable elution method known in the art.

### Samples

In some embodiments, nucleic acids (e.g., DNA or RNA) are isolated from a biological sample containing a variety of other components, such as proteins, lipids, and other (e.g., non-target or non-template) nucleic acids. Nucleic acid molecules can be obtained from any material (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present technology include viral particles or preparations thereof. In some embodiments a nucleic acid is isolated from a sample for use as a template in an amplification reaction (e.g., to prepare an amplicon library or fragment library for sequencing). In some embodiments a nucleic acid is isolated from a sample for use in preparing a library of fragments.

Nucleic acid molecules can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), and/or other specimens.

Any tissue or body fluid specimen may be used as a source for nucleic acid for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Nucleic acid molecules can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281).

In some embodiments, the technology provides for the size selection of nucleic acids, e.g., to remove very short fragments or very long fragments. In various embodiments, the size is limited to be 0.5, 1, 2, 3, 4, 5, 7, 10, 12, 15, 20, 25, 30, 50, 100 kb or kbp or longer.

In various embodiments, a nucleic acid is amplified. Any amplification method known in the art may be used. Examples of amplification techniques that can be used include, but are not limited to, PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), hot start PCR, nested PCR, in situ polony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid based sequence amplification (NABSA). Other amplification methods that can be used herein include those described in U.S. Pat. Nos. 5,242,794; 5,494,810; 4,988,617; and 6,582,938.

In some embodiments, end repair is performed to generate blunt end 5′ phosphorylated nucleic acid ends using commercial kits, such as those available from Epicentre Biotechnologies (Madison, Wis.).

In some embodiments, the technology finds use in normalizing an amplicon panel, e.g., an amplicon panel library. An amplicon panel is a collection of amplicons that are related, e.g., to a disease (e.g., a polygenic disease), disease progression, developmental defect, constitutional disease (e.g., a state having an etiology that depends on genetic factors, e.g., a heritable (non-neoplastic) abnormality or disease), metabolic pathway, pharmacogenomic characterization, trait, organism (e.g., for species identification), group of organisms, geographic location, organ, tissue, sample, environment (e.g., for metagenomic and/or ribosomal RNA (e.g., ribosomal small subunit (SSU), ribosomal large subunit (LSU), 5S, 16S, 18S, 23S, 28S, internal transcribed sequence (ITS) rRNA) studies), gene, chromosome, etc. For example, a cancer panel comprises specific genes or mutations in genes that have established relevancy to a particular cancer phenotype (e.g., one or more of ABL1, AKT1, AKT2, ATM, PDGFRA, EGFR, FGFR (e.g., FGFR1, FGFR2, FGFR3), BRAF (e.g., comprising a mutation at V600, e.g., a V600E mutation), RUNX1, TET2, CBL, EGFR, FLT3, JAK2, JAK3, KIT, RAS (e.g., KRAS (e.g., comprising a mutation at G12, G13, or A146, e.g., a G12A, G12S, G12C, G12D, G13D, or A146T mutation), HRAS (e.g., comprising a mutation at G12, e.g., a G12V mutation), NRAS (e.g., comprising a mutation at Q61, e.g., a Q61R or Q61K mutation)), MET, PIK3CA (e.g., comprising a mutation at H1047, e.g., a H1047L, H1047L, or H1047R mutation), PTEN, TP53 (e.g., comprising a mutation at R248, Y126, G245, or A159, e.g., a R248W, G245S, or A159D mutation), VEGFA, BRCA, RET, PTPN11, HNHF1A, RB1, CDH1, ERBB2, ERBB4, SMAD4, SKT11 (e.g., comprising a mutation at Q37), ALK, IDH1, IDH2, SRC, GNAS, SMARCB1, VHL, MLH1, CTNNB1, KDR, FBXW7, APC, CSF1R, NPM1, MPL, SMO, CDKN2A, NOTCH1, CDK4, CEBPA, CREBBP, DNMT3A, FES, FOXL2, GATA1, GNA11, GNAQ, HIF1A, IKBKB, MEN1, NF2, PAX5, PIK3R1, PTCH1, STK11, etc.). Some amplicon panels are directed toward particular “cancer hotspots”, that is, regions of the genome containing known mutations that correlate with cancer progression and therapeutic resistance.

In some embodiments, an amplicon panel for a single gene includes amplicons for the exons of the gene (e.g., 1, 2, 3, 4, 5, 6, 7, 8, 9, 10, 11, 12, 13, 14, 15, 16, 17, 18, 19, 20, or more exons). In some embodiments, an amplicon panel for species (or strain, sub-species, type, sub-type, genus, or other taxonomic level and/or operational taxonomic unit (OTU) based on a measure of phylogenetic distance) identification may include amplicons corresponding to a suite of genes or loci that collectively provide a specific identification of one or more species (or strain, sub-species, type, sub-type, genus, or other taxonomic level) relative to other species (or strain, sub-species, type, sub-type, genus, or other taxonomic level) (e.g., for bacteria (e.g., MRSA), viruses (e.g., HIV, HCV, HBV, respiratory viruses, etc.)) or that are used to determine drug resistance(s) and/or sensitivity/ies (e.g., for bacteria (e.g., MRSA), viruses (e.g., HIV, HCV, HBV, respiratory viruses, etc.)).

The amplicons of the panel typically comprise 100 to 1000 base pairs, e.g., in some embodiments the amplicons of the panel comprise approximately 100, 125, 150, 175, 200, 225, 250, 275, 300, 325, 350, 375, 400, 325, 350, 375, 400, 425, 450, 475, 500, 525, 550, 575, 600, 625, 650, 675, 700, 725, 750, 775, 800, 825, 850, 875, 900, 925, 950, 975, or 1000 base pairs. In some embodiments, an amplicon panel comprises a collection of amplicons that span a genome, e.g., to provide a genome sequence.

The amplicon panel is often produced through use of amplification oligonucleotides (e.g., to produce the amplicon panel from the sample) and/or oligonucleotide probes for sequencing disease-related genes, e.g., to assess the presence of particular mutations and/or alleles in the genome. In some embodiments, 10, 20, 30, 40, 50, 60, 70, 80, 90, 100, 150, 200, 300, 400, 500, 1000, or more genes, loci, regions, etc. are targeted to produce, e.g., 10, 20, 30, 40, 50, 60, 70, 80, 90, 100, 150, 200, 300, 400, 500, 1000, or more amplicons. In some embodiments, the amplicons are produced in a highly multiplexed, single tube amplification reaction. In some embodiments, the amplicons are produced in a collection of singleplex amplification reactions (e.g., 10 to 100, 100 to 1000, or 1000 or more reactions). In some embodiments, the collection of singleplex amplification reactions are pooled. In some embodiments, the singleplex amplification reactions are performed in parallel.

In some preferred embodiments, a number of amplification (e.g., thermal) cycles is minimized (e.g., in some embodiments, less than the number of cycles used in conventional technologies) to retain uniform coverage of target sequences by the amplicons, to provide accurate representation of target sequences in the amplicons, and/or to minimize and/or eliminate bias such as the bias introduced into amplified samples during the middle and late stages of amplification. Accordingly, the amount of DNA (e.g., amplicons) produced is less than that used as input for conventional normalization technologies. In some embodiments, the amount of amplicon DNA used as input to the normalization technology provided herein is less than 250 ng; in some embodiments, the amount of amplicon DNA used as input to the normalization technology provided herein is less than 100 ng. And, in some embodiments, the number of amplicons in the sample used as input to the normalization technology provided herein is less than 200, less than 150, less than 100, e.g., 1 to 150 amplicons. As such, the technology finds use in processing amplicon libraries comprising low (e.g., in mass and/or in number) amounts of amplicons to prepare samples for a next-generation sequencing workflow.

Production of an amplicon panel is often associated with downstream next-generation sequencing to obtain the sequences of the amplicons of the panel. That is, the amplification is used to target the genome and provide selected regions of interest for NGS. This target enrichment focuses sequencing efforts to specific regions of a genome, thus providing a more cost-effective alternative to sequencing an entire genome and providing increased depth of coverage at the regions of interest (e.g., for improved detection of rare variation and/or lower rates of false negatives and/or false positives). Moreover, NGS provides a technology for targeting multiple amplicons in a single test.

### Nucleic Acid Sequencing

In some embodiments of the technology, nucleic acid sequence data are generated. Various embodiments of nucleic acid sequencing platforms (e.g., a nucleic acid sequencer) include components as described below. According to various embodiments, a sequencing instrument includes a fluidic delivery and control unit, a sample processing unit, a signal detection unit, and a data acquisition, analysis, and control unit. Various embodiments of the instrument provide for automated sequencing that is used to gather sequence information from a plurality of sequences in parallel and/or substantially simultaneously.

In some embodiments, the fluidics delivery and control unit includes a reagent delivery system. The reagent delivery system includes a reagent reservoir for the storage of various reagents. The reagents can include RNA-based primers, forward/reverse DNA primers, nucleotide mixtures (e.g., in some embodiments, compositions comprise nucleotide analogs) for sequencing-by-synthesis, buffers, wash reagents, blocking reagents, stripping reagents, and the like. Additionally, the reagent delivery system can include a pipetting system or a continuous flow system that connects the sample processing unit with the reagent reservoir.

In some embodiments, the sample processing unit includes a sample chamber, such as flow cell, a substrate, a micro-array, a multi-well tray, or the like. The sample processing unit can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber. In some embodiments, the signal detection unit can include an imaging or detection sensor. For example, the imaging or detection sensor (e.g., a fluorescence detector or an electrical detector) can include a CCD, a CMOS, an ion sensor, such as an ion sensitive layer overlying a CMOS, a current detector, or the like. The signal detection unit can include an excitation system to cause a probe, such as a fluorescent dye, to emit a signal. The detection system can include an illumination source, such as an arc lamp, a laser, a light emitting diode (LED), or the like. In particular embodiments, the signal detection unit includes optics for the transmission of light from an illumination source to the sample or from the sample to the imaging or detection sensor. Alternatively, the signal detection unit may not include an illumination source, such as for example, when a signal is produced spontaneously as a result of a sequencing reaction. For example, a signal can be produced by the interaction of a released moiety, such as a released ion interacting with an ion-sensitive layer, or a pyrophosphate reacting with an enzyme or other catalyst to produce a chemiluminescent signal. In another example, changes in an electrical current, voltage, or resistance are detected without the need for an illumination source.

In some embodiments, a data acquisition analysis and control unit monitors various system parameters. The system parameters can include temperatures of various portions of the instrument, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of the instruments and systems are used to practice sequencing methods such as sequencing by synthesis, single molecule methods, and other sequencing techniques. Sequencing by synthesis can include the incorporation of dye labeled nucleotides, chain termination, ion/proton sequencing, pyrophosphate sequencing, or the like. Single molecule techniques can include staggered sequencing, where the sequencing reaction is paused to determine the identity of the incorporated nucleotide.

In some embodiments, the sequencing instrument determines the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In some embodiments, the nucleic acid can include or be derived from a fragment library, an amplicon library, a mate pair library, a ChIP fragment, or the like. In particular embodiments, the sequencing instrument can obtain the sequence information from a single nucleic acid molecule or from a group of substantially identical nucleic acid molecules.

In some embodiments, the sequencing instrument can output nucleic acid sequencing read data in a variety of different output data file types/formats, including, but not limited to: *.txt, *.fasta, *.csfasta, *seq.txt, *qseq.txt, *.fastq, *.sff, *prb.txt, *.sms, *srs, and/or *.qv.

### Next-Generation Sequencing Technologies

Particular sequencing technologies contemplated by the technology are next-generation sequencing (NGS) methods that share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), the NGS fragment library is clonally amplified in-situ by capturing single template molecules with beads bearing oligonucleotides complementary to the adapters. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and a luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, the fragments or amplicons of the NGS library are captured on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 100 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves clonal amplification of the NGS fragment library by emulsion PCR. Following this, beads bearing template are immobilized on a derivatized surface of a glass flow-cell, and a primer complementary to the adapter oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, HeliScope by Helicos BioSciences is employed (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). HeliScope equencing is achieved by addition of polymerase and serial addition of fluorescently-labeled dNTP reagents. Incorporation events result in a fluor signal corresponding to the dNTP, and signal is captured by a CCD camera before each round of dNTP addition. Sequence read length ranges from 25-50 nucleotides, with overall output exceeding 1 billion nucleotide pairs per analytical run.

In some embodiments, 454 sequencing by Roche is used (Margulies et al. (2005) Nature 437: 376-380). 454 sequencing involves two steps. In the first step, DNA is sheared into fragments of approximately 300-800 base pairs and the fragments are blunt ended. Oligonucleotide adapters are then ligated to the ends of the fragments. The adapters serve as primers for amplification and sequencing of the fragments. The fragments can be attached to DNA capture beads, e.g., streptavidin-coated beads using, e.g., an adapter that contains a 5′-biotin tag. The fragments attached to the beads are PCR amplified within droplets of an oil-water emulsion. The result is multiple copies of clonally amplified DNA fragments on each bead. In the second step, the beads are captured in wells (picoliter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a fragment of the NGS library to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers the ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs. However, the cost of acquiring a pH-mediated sequencer is approximately $50,000, excluding sample preparation equipment and a server for data analysis.

Another exemplary nucleic acid sequencing approach that may be adapted for use with the present technology was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “HIGH THROUGHPUT NUCLEIC ACID SEQUENCING BY EXPANSION,” filed Jun. 19, 2008, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which fragments of the NGS library are immobilized, primed, then subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10⁻²¹ liters). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters. At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations that promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

In some embodiments, nanopore sequencing is used (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

In some embodiments, a sequencing technique uses a chemical-sensitive field effect transistor (chemFET) array to sequence DNA (for example, as described in US Patent Application Publication No. 20090026082). In one example of the technique, DNA molecules are placed into reaction chambers, and the template molecules are hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, sequencing technique uses an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

In some embodiments, “four-color sequencing by synthesis using cleavable fluorescent nucleotide reversible terminators” as described in Turro, et al. PNAS 103: 19635-40 (2006) is used, e.g., as commercialized by Intelligent Bio-Systems. The technology described in U.S. Pat. Appl. Pub. Nos. 2010/0323350, 2010/0063743, 2010/0159531, 20100035253, 20100152050, incorporated herein by reference for all purposes.

Processes and systems for such real time sequencing that may be adapted for use with the technology are described in, for example, U.S. Pat. No. 7,405,281, entitled “Fluorescent nucleotide analogs and uses therefor”, issued Jul. 29, 2008 to Xu et al.; U.S. Pat. No. 7,315,019, entitled “Arrays of optical confinements and uses thereof”, issued Jan. 1, 2008 to Turner et al.; U.S. Pat. No. 7,313,308, entitled “Optical analysis of molecules”, issued Dec. 25, 2007 to Turner et al.; U.S. Pat. No. 7,302,146, entitled “Apparatus and method for analysis of molecules”, issued Nov. 27, 2007 to Turner et al.; and U.S. Pat. No. 7,170,050, entitled “Apparatus and methods for optical analysis of molecules”, issued Jan. 30, 2007 to Turner et al.; and U.S. Pat. Pub. Nos. 20080212960, entitled “Methods and systems for simultaneous real-time monitoring of optical signals from multiple sources”, filed Oct. 26, 2007 by Lundquist et al.; 20080206764, entitled “Flowcell system for single molecule detection”, filed Oct. 26, 2007 by Williams et al.; 20080199932, entitled “Active surface coupled polymerases”, filed Oct. 26, 2007 by Hanzel et al.; 20080199874, entitled “CONTROLLABLE STRAND SCISSION OF MINI CIRCLE DNA”, filed Feb. 11, 2008 by Otto et al.; 20080176769, entitled “Articles having localized molecules disposed thereon and methods of producing same”, filed Oct. 26, 2007 by Rank et al.; 20080176316, entitled “Mitigation of photodamage in analytical reactions”, filed Oct. 31, 2007 by Eid et al.; 20080176241, entitled “Mitigation of photodamage in analytical reactions”, filed Oct. 31, 2007 by Eid et al.; 20080165346, entitled “Methods and systems for simultaneous real-time monitoring of optical signals from multiple sources”, filed Oct. 26, 2007 by Lundquist et al.; 20080160531, entitled “Uniform surfaces for hybrid material substrates and methods for making and using same”, filed Oct. 31, 2007 by Korlach; 20080157005, entitled “Methods and systems for simultaneous real-time monitoring of optical signals from multiple sources”, filed Oct. 26, 2007 by Lundquist et al.; 20080153100, entitled “Articles having localized molecules disposed thereon and methods of producing same”, filed Oct. 31, 2007 by Rank et al.; 20080153095, entitled “CHARGE SWITCH NUCLEOTIDES”, filed Oct. 26, 2007 by Williams et al.; 20080152281, entitled “Substrates, systems and methods for analyzing materials”, filed Oct. 31, 2007 by Lundquist et al.; 20080152280, entitled “Substrates, systems and methods for analyzing materials”, filed Oct. 31, 2007 by Lundquist et al.; 20080145278, entitled “Uniform surfaces for hybrid material substrates and methods for making and using same”, filed Oct. 31, 2007 by Korlach; 20080128627, entitled “SUBSTRATES, SYSTEMS AND METHODS FOR ANALYZING MATERIALS”, filed Aug. 31, 2007 by Lundquist et al.; 20080108082, entitled “Polymerase enzymes and reagents for enhanced nucleic acid sequencing”, filed Oct. 22, 2007 by Rank et al.; 20080095488, entitled “SUBSTRATES FOR PERFORMING ANALYTICAL REACTIONS”, filed Jun. 11, 2007 by Foquet et al.; 20080080059, entitled “MODULAR OPTICAL COMPONENTS AND SYSTEMS INCORPORATING SAME”, filed Sep. 27, 2007 by Dixon et al.; 20080050747, entitled “Articles having localized molecules disposed thereon and methods of producing and using same”, filed Aug. 14, 2007 by Korlach et al.; 20080032301, entitled “Articles having localized molecules disposed thereon and methods of producing same”, filed Mar. 29, 2007 by Rank et al.; 20080030628, entitled “Methods and systems for simultaneous real-time monitoring of optical signals from multiple sources”, filed Feb. 9, 2007 by Lundquist et al.; 20080009007, entitled “CONTROLLED INITIATION OF PRIMER EXTENSION”, filed Jun. 15, 2007 by Lyle et al.; 20070238679, entitled “Articles having localized molecules disposed thereon and methods of producing same”, filed Mar. 30, 2006 by Rank et al.; 20070231804, entitled “Methods, systems and compositions for monitoring enzyme activity and applications thereof”, filed Mar. 31, 2006 by Korlach et al.; 20070206187, entitled “Methods and systems for simultaneous real-time monitoring of optical signals from multiple sources”, filed Feb. 9, 2007 by Lundquist et al.; 20070196846, entitled “Polymerases for nucleotide analog incorporation”, filed Dec. 21, 2006 by Hanzel et al.; 20070188750, entitled “Methods and systems for simultaneous real-time monitoring of optical signals from multiple sources”, filed Jul. 7, 2006 by Lundquist et al.; 20070161017, entitled “MITIGATION OF PHOTODAMAGE IN ANALYTICAL REACTIONS”, filed Dec. 1, 2006 by Eid et al.; 20070141598, entitled “Nucleotide Compositions and Uses Thereof”, filed Nov. 3, 2006 by Turner et al.; 20070134128, entitled “Uniform surfaces for hybrid material substrate and methods for making and using same”, filed Nov. 27, 2006 by Korlach; 20070128133, entitled “Mitigation of photodamage in analytical reactions”, filed Dec. 2, 2005 by Eid et al.; 20070077564, entitled “Reactive surfaces, substrates and methods of producing same”, filed Sep. 30, 2005 by Roitman et al.; 20070072196, entitled “Fluorescent nucleotide analogs and uses therefore”, filed Sep. 29, 2005 by Xu et al; and 20070036511, entitled “Methods and systems for monitoring multiple optical signals from a single source”, filed Aug. 11, 2005 by Lundquist et al.; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

In some embodiments, the quality of data produced by a next-generation sequencing platform depends on the concentration of DNA (e.g., an NGS library such as a fragment library or an amplicon panel library) that is loaded onto the sequencer workflow clonal amplification step. For instance, loading a concentration that is below a minimal threshold may result in low or sub-optimal sequencer output while loading a concentration that is above a maximum threshold may result in low quality sequence or no sequencer output. Accordingly, the technology provided herein finds use in preparing a sample having an appropriate concentration for sequencing, e.g., such that the sequence data that is output has a desirable quality.

### Uses

The technology is not limited to particular uses, but finds use in a wide range of research (basic and applied), clinical, medical, and other biological, biochemical, and molecular biological applications. The technology finds use in methods, kits, systems, etc. that are associated with providing a sample of nucleic acid that is concentration normalized. Some exemplary uses of the technology include genetics, genomics, and/or genotyping, e.g., of plants, animals, and other organisms, e.g., to identify haplotypes, phasing, and/or linkage of mutations and/or alleles. In some embodiments, the technology finds use in sequencing related to cancer diagnosis, treatment, and therapy.

In addition, the technology finds use in the field of infectious disease, e.g., in identifying infectious agents such as viruses, bacteria, fungi, etc., and in determining viral types, families, species, and/or quasi-species, and to identify haplotypes, phasing, and/or linkage of mutations and/or alleles. Other particular and non-limiting illustrative examples in the area of infectious disease include characterizing antibiotic resistance determinants; tracking infectious organisms for epidemiology; monitoring the emergence and evolution of resistance mechanisms; identifying species, sub-species, strains, extra-chromosomal elements, types, etc. associated with virulence, monitoring the progress of treatments, etc.

In some embodiments, the technology finds use in transplant medicine, e.g., for typing of the major histocompatibility complex (MHC), typing of the human leukocyte antigen (HLA), and for identifying haplotypes, phasing, and/or linkage of mutations and/or alleles associated with transplant medicine (e.g., to identify compatible donors for a particular host needing a transplant, to predict the chance of rejection, to monitor rejection, to archive transplant material, for medical informatics databases, etc.).

In some embodiments, the technology finds use in oncology and fields related to oncology. Particular and non-limiting illustrative examples in the area of oncology are detecting genetic and/or genomic aberrations related to cancer, predisposition to cancer, and/or treatment of cancer. For example, in some embodiments the technology finds use in detecting the presence of a mutation, polymorphism, allele, or a chromosomal translocation associated with cancer. In some embodiments, the technology finds use in cancer screening, cancer diagnosis, cancer prognosis, measuring minimal residual disease, and selecting and/or monitoring a course of treatment for a cancer.

Some embodiments comprise use of a computer (e.g., a microchip) that executes computer instructions to analyze sequencing data and present results (e.g., to a user).

### Kits

The present technology also provides embodiments of kits. In one embodiment, a kit comprises a solid phase carrier (e.g., as a solution, slurry, powder for resuspension, etc.) and a buffer. Kits, in some embodiments, further comprise additional buffers (e.g., wash buffers and/or elution buffers); enzymes for nucleic acid degradation, ligation, end finishing, etc.; nucleotides, and instructions for use. In particular embodiments, the kit comprises magnetic microparticles comprising COOH groups and, in some embodiments, magnetic microparticles comprising an oligo dT group or a derivative thereof.

Although the disclosure herein refers to certain illustrated embodiments, it is to be understood that these embodiments are presented by way of example and not by way of limitation.

### EXAMPLES

### Example 1

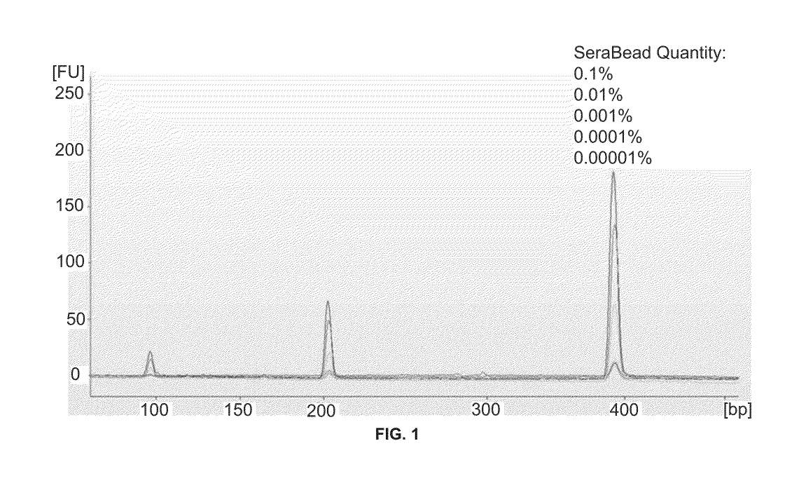

First, data collected during experiments conducted during the development of embodiments of the technology described herein indicated that COOH bead quantity (e.g., % bead solids) limits the quantity of DNA recovered. In particular, a bead quantity titration experiment was performed with both Agentcourt Ampure XP beads and 1-μm Sera-Mag beads (Fisher Scientific, cat#09-981-123) on a fixed amount of DNA ladder (FIG. 1). The data showed decreasing recovery of approximately 100, 200, and 400 base pair fragments with decreasing quantities of beads (0.1 to 0.00001% beads; see FIG. 1).

### Example 2

Next, during the development of embodiments of the technology provided herein, experiments were performed to test methods and formulations to concentration normalize a NGS multiplex amplicon panel pool (Abbott Molecular).

### Materials and Methods

Carboxylated beads were 8-μm magnetic beads functionalized with COOH (Bangs Laboratories, Inc. “COMPEL™ Magnetic COOH modified, 8 μm (5% solid), catalogue number UMC4N/10487). The bead buffer comprised PEG, NaCl, Tris (pH 7), EDTA, Tween-20, and water. An exemplary bead buffer composition comprises ingredients in the following concentrations:

Bead Buffer Recipe

| | Volume (uL) | Final Concentration | Final Units |
|---|---|---|---|
| 40% PEG 8000 | 1500 | 20 | % |
| in dH2O | | | |
| 5M NaCl | 300 | 0.5 | M |
| 1M Tris (pH 7) | 1180 | 0.393 | M |
| 0.5M EDTA | 6 | 0.001 | M |
| 10% Tween20 | 14 | 0.047 | % |
| Total | 3000 | | |

Samples for testing included amplicon products produced using a 20-plex multiplex PCR reaction (Abbott Molecular) and 10 ng of Human Placental gDNA template. The following dilution series of samples was tested:

Sampler Undiluted

Sample2: 1:3 dilution of Sample1

Sample3: 1:3 dilution of Sample2

The bead mix was produced as follows: 8-μm magnetic beads functionalized with COOH are washed with molecular biology grade H₂O (e.g., the same volume of H₂O is used as the amount of bead mix aliquoted so that the percentage of solids is 5% after wash and resuspension in fresh H₂O).

Beads were washed by aliquoting the desired about of beads into a non-stick 1.5 mL microcentrifuge tube and placing the tube on a magnetic stand for 2 minutes or until solution becomes clear. Then, the tube is washed while in the magnetic stand and the supernatant is carefully removed. Beads are dried by leaving the tube cap open and leaving the tube at room temperature for 5 minutes. The tube with beads is taken off the magnet and beads are resuspended in molecular biology grade H₂O using the same volume of H₂O as the original volume of bead mix used.

Then, the bead buffer is mixed with the washed beads. For example, for every 98 μl of bead buffer, add 2 μl of washed and resuspended beads (e.g., to make 500 μl of normalization bead mixture add 490 μl of buffer and 10 μl of resuspended bead mix). Bead mix can be stored at 4° C. until use.

Simultaneous size selection, purification, and concentration normalization was performed according to the following protocol. Prior to beginning the procedure, fresh 60% EtOH (500 μL per sample) was mixed. Then, 1 part of sample and 2 parts of bead mix are combined and mixed in a 1.5 mL non-stick microcentrifuge tube (e.g., 25 μl of sample+50 μl of bead mix). The sample is mixed well (e.g., by gentle vortex) and incubated at room temperature for 5 minutes. Next, the tube is placed in a magnet rack (e.g., for 2 minutes or until the solution becomes clear). The supernatant is carefully removed while the tube is still placed in the magnetic rack. Beads are washed, e.g., two times with 200 μl of 60% EtOH, while the tube remains in the rack. Beads are then dried in air for 5 minutes and the tube is removed from the rack. Beads are resuspended in an appropriate elution volume using low-TE buffer. The resuspension is placed in the rack for 1 minute after which the supernatant is removed without disturbing the bead pellet.

Further, during the development of embodiments of the technology provided herein, experiments were conducted to demonstrate size, purification, and concentration normalization in a single step. In particular, experiments were conducted using Sample 1, Sample 2, and Sample 3 (e.g., a 3-fold dilution series) comprising a multiplex amplicon library to determine buffer component concentrations appropriate for concentration normalization, sizing, and purification of NGS libraries.

### Results

After showing in Example 1 that the amount of DNA recovered is limited by the quantity of beads used in the recovery procedure, experiments were performed to normalize a varying range input amounts of DNA to the same final concentration using 1 μm Sera-Mag COOH beads. However, using the 1-μm COOH beads, concentration normalization was not produced across a satisfactory range of DNA input amounts.

Next, experiments were conducted using larger 8-μm COOH beads (e.g., having a lower surface area per unit mass and thus a lower binding capacity per unit mass) from Bangs Laboratories, Inc. The 8-μm beads provided simultaneous purification, size selection, and concentration normalization of a multiplex amplicon library across the 3-fold multiplex amplicon sample dilution series mentioned above (e.g., Sample 1, Sample 2, and Sample 3).

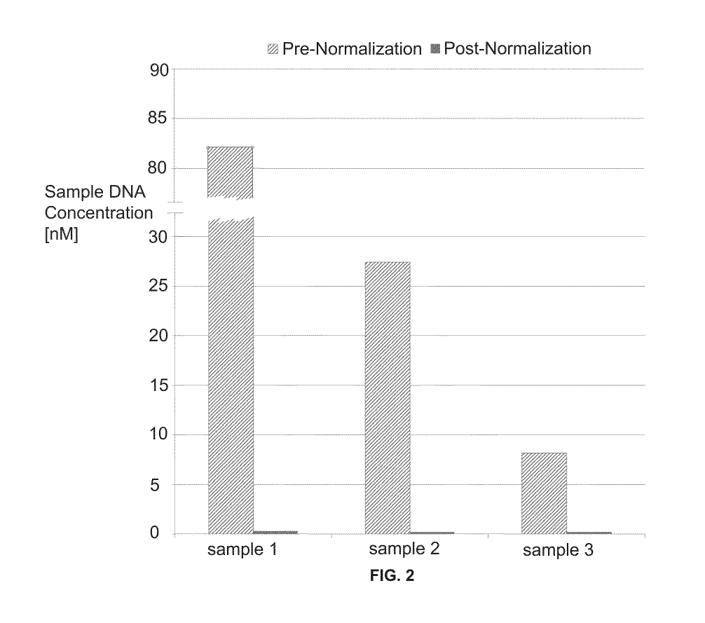

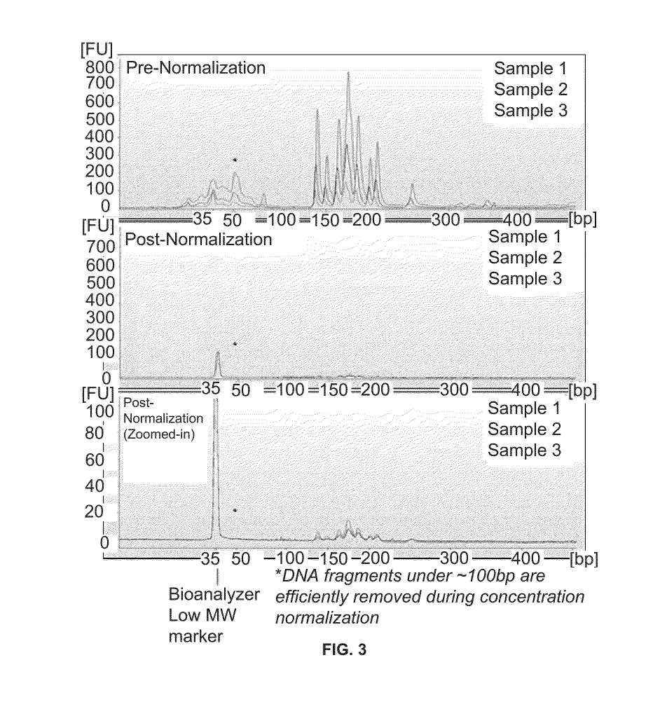

In these experiments, the 3 independent NGS amplicon libraries having concentrations ranging from approximately 8 nM to approximately 80 nM were used as input to the technology. After concentration normalization according to the technology provided, the concentrations of the libraries were uniformly approximately 0.2 nM to 0.3 nM (FIG. 2). In addition, embodiments of the methods provided a purification and size selection of the samples by efficiently removing enzymes, dNTPs, salts, and fragments of nucleic acids under approximately 100 base pairs (FIG. 3). Fragment size analysis and quantification were performed on an Agilent Bioanalyzer 2100.

The data from these experiments showed that application of the technology to the NGS multiplex amplicon library produced a purified and concentration normalized NGS amplicon pool that is ready for loading into an NGS platform workflow.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in the art are intended to be within the scope of the following claims.

1-31. (canceled)

32. A method for normalizing the concentration of a next generation sequencing (NGS) library, the method comprising:
a) mixing:
1) an input next-generation sequencing library comprising a first amount of nucleic acids with
2) a capture substrate having a capacity to bind a second amount of nucleic acids that is less than the first amount of nucleic acids to provide a capture mixture comprising unbound nucleic acids and a capture substrate comprising bound nucleic acids; and
b) eluting the bound nucleic acids from the capture substrate to provide as output a concentration normalized NGS library.

33. The method of claim 32 further comprising:
a) binding nucleic acids to the capture substrate;
b) removing unbound nucleic acids from the capture mixture; and/or
c) washing the capture substrate comprising the bound nucleic acids.

34. The method of claim 32 wherein the capture substrate comprises a paramagnetic microparticle functionalized with a carboxyl group.

35. The method of claim 32 wherein the ratio of the first amount of nucleic acids to the second amount of nucleic acids is more than 1000, more than 100, or more than 10.

36. The method of claim 32 further comprising:
a) ligating an adapter to a nucleic acid;
b) adding a nucleic acid precipitating reagent; and/or
c) loading the concentration normalized NGS library into a next generation sequencer work flow.

37. The method of claim 32 further comprising size-selecting the NGS library by adjusting buffer components or adjusting ionic strength.

38. The method of claim 32 further comprising combining two or more concentration normalized NGS libraries to provide a multiplex concentration normalized NGS library.

39. The method of claim 32 wherein the concentration normalized NGS library comprises:
a) nucleic acids at a concentration of less than 1 nM, less than 0.75 nM, less than 0.55 nM, less than 0.25 nM, less than 0.1 nM, or less than 0.05 nM;
b) nucleic acids that comprise more than 100 bp; and/or
c) less than 200, less than 150, less than 100, less than 50, less than 25, less than 10, less than 5 nucleic acids.

40. The method of claim 32 wherein the input NGS library comprises less than 250 ng, less than 200, less than 150, or less than 100 ng of nucleic acid.

41. The method of claim 32 wherein the steps of the method are performed in a single vessel.

42. The method of claim 32 wherein the input NGS library is an amplicon panel library or a fragment library.

43. A concentration normalized NGS library or a concentration normalized amplicon panel library produced by a method according to claim 32

44. A method for simultaneous size selection, purification, and concentration normalization of a DNA amplicon library, the method comprising:
a) mixing a sample comprising a DNA amplicon library with a solution comprising PEG, NaCl, and magnetic beads functionalized with carboxylate groups;
b) washing the beads with EtOH; and
c) eluting the DNA amplicon library from the beads to prepare a size selected, purified, and concentration normalized DNA amplicon library ready for input to a NGS workflow.

45. The method of claim 44 wherein the sample comprising a DNA amplicon library and the solution are mixed in a 1:2 ratio.

46. The method of claim 44 wherein the solution comprises 20% PEG 8000, 0.5 M NaCl, and 8-μm magnetic beads at 5% w/v beads/solution.

47. The method of claim 44 wherein the concentration normalized DNA amplicon library comprises a concentration of DNA that is 0.2 nM to 0.3 nM.

48. The method of claim 44 wherein the concentration normalized DNA amplicon library comprises DNA that is greater than approximately 100 base pairs.

49. The method of claim 44 comprising washing with 60% EtOH.

50. The method of claim 44 further comprising loading the concentration normalized NGS library into a next generation sequencer work flow.
